(19) United States
(12) Patent Application Publication
(10) Pub. No.: US 2023/0338911 A1
(43) Pub. Date: Oct. 26, 2023

(54) HIGH TEMPERATURE AIR SEPARATION MODULE FOR AN ODH COMPLEX

(71) Applicant: NOVA CHEMICALS (INTERNATIONAL) S.A., Fribourg (CH)

(72) Inventors: Vasily SIMANZHENKOV, Calgary (CA); Bolaji OLAYIWOLA, Calgary (CA); Shahin GOODARZNIA, Calgary (CA)

(21) Appl. No.: 18/030,164

(22) PCT Filed: Oct. 6, 2021
(86) PCT No.: PCT/IB2021/059183

§ 371 Date: Apr. 4, 2023

Related U.S. Application Data
(60) Provisional application No. 63/089,076, filed on Oct. 8, 2020.

(51) Int. Cl. B01J 8/02; B01J 27/057; B01D 53/14; B01D 53/62; B01D 53/78; B01D 3/14; B01D 53/22

(52) U.S. Cl. CPC B01J 8/0278; B01J 27/0576; B01D 53/1406; B01D 53/1475; B01D 53/62; B01D 53/78; B01D 3/143; B01D 53/22; B01D 2257/504; B01D 2257/80; B01D 2257/104; B01D 2258/0283; B01D 2252/204

(57) ABSTRACT

A chemical complex to perform oxidative dehydrogenation of C2-C4 alkanes, to C2-C4 alkenes, the chemical complex involving at least one oxidative dehydrogenation reactor containing one or more mixed metal oxide catalysts and designed to accept, optionally in the presence of a heat removal diluent gas, an oxygen containing gas and a C2-C4 alkane containing gas, and to produce a product stream including a corresponding C2-C4 alkene and one or more of: an unreacted C2-C4 alkane; oxygen; heat removal diluent gas; carbon oxides, including carbon dioxide and carbon monoxide; oxygenates, including but not limited to, one or more of acetic acid, acrylic acid and maleic acid; and water; and involving a combustion chamber for combusting a product stream and at least one fuel stream and optionally at least one stream including oxygen, the combustion chamber producing a flue gas at a temperature of 850° C. to 1500° C.

### CLAIM OF PRIORITY

This application claims priority to U.S. Provisional Application No. 63/089,076 filed on Oct. 8, 2020, the entire contents of which are hereby incorporated by reference.

### TECHNICAL FIELD

The present disclosure relates generally to oxidative dehydrogenation (ODH) of lower alkanes into corresponding alkenes. More specifically, the present disclosure relates to a chemical complex for ODH that includes an oxygen separation module, and the use of a hot air separation membrane, whereby the energy to the membrane is supplied by an external combustion device.

### BACKGROUND ART

Catalytic oxidative dehydrogenation (ODH) of alkanes into corresponding alkenes is an alternative to steam cracking; steam cracking is the method of choice for the majority of today's commercial-scale producers. Despite its widespread use, steam cracking has its downsides. Steam cracking is energy intensive, requiring temperatures in the range of 700° C. to 1000° C. to satisfy the highly endothermic nature of the cracking reactions. The process is expensive owing to the high fuel demand, the requirement for reactor materials that can withstand the high temperatures, and the necessity for separation of unwanted by-products using downstream separation units. The production of coke by-product requires periodic shutdown for cleaning and maintenance. For ethylene producers, the selectivity for ethylene is only around 80-85 mol % for a conversion rate that does not generally exceed 60%. In contrast, ODH operates at lower temperatures, does not produce coke, and using newer-developed catalysts provides selectivity for ethylene of around 98 mol % at close to 60% conversion.

### SUMMARY OF INVENTION

This disclosure relates to a chemical complex for ODH that includes an oxygen separation module with energy supplied by hot feed gas, the energy is a result of combustion in a device external to the oxygen transport membrane.

Provided in this disclosure is a chemical complex for oxidative dehydrogenation of C2-C4 alkanes, the chemical complex including: at least one oxidative dehydrogenation reactor, including a mixed metal oxide catalyst and designed to accept, optionally in the presence of a heat removal diluent gas, an oxygen containing gas and a C2-C4 alkane containing gas, and to produce a product stream including a corresponding C2-C4 alkene and one or more of: an unreacted C2-C4 alkane; oxygen; heat removal diluent gas; carbon oxides, including carbon dioxide and carbon monoxide; oxygenates, including but not limited to, one or more of acetic acid, acrylic acid and maleic acid; and water; a quench tower for quenching the product stream and for removing water and soluble oxygenates from the product stream; an amine wash for removing carbon dioxide from the product stream; a dryer for removal of water from the product stream; a distillation tower for removing C2/C2+ hydrocarbons from the product stream to produce an overhead stream enriched with C1 hydrocarbons and any other compounds lighter than C2/C2+ hydrocarbons; a combustion chamber for combusting the overhead stream and at least one fuel stream and optionally at least one stream including oxygen, the combustion chamber producing a flue gas at a temperature of 850° C. to 1500° C., the heat from the fuel gas used to heat an oxygen separation module either directly (as shown in FIG. 1) or indirectly (as shown in FIG. 2) by heating the air and/or sweep gas to the membrane; an oxygen separation module including: an oxygen transport membrane housed inside a sealed vessel and having a retentate side and a permeate side; a first inlet for introducing the overhead stream, combustible fuel, or both into the retentate side; a second inlet for introducing the overhead stream, combustible fuel, or both into the permeate side; an air inlet for introducing air into the retentate side; an exhaust stream for discharge of oxygen depleted air and combustion products from the retentate side; an outlet stream for removing oxygen enriched gas and combustion products from the permeate side; wherein the oxygen enriched gas from the permeate side is directed back to the oxidative dehydrogenation reactor as or part of the oxygen containing gas introduced into the at least one oxidative dehydrogenation reactor.

In some embodiments, the chemical complex includes an outlet stream for removing oxygen enriched gas and combustion products from the permeate side, at least part of the outlet stream feeding a combustion chamber, at least part of the flue gas from the combustion chamber supplying heat to the oxygen separation module, such that the temperature of the oxygen transport membrane is from about 850° C. to 1500° C. In some embodiments, the chemical complex includes at least part of the flue gas from the combustion chamber recycled to the oxygen separation module supplying heat, such that the temperature of the oxygen transport membrane is 850° C. to 1500° C.

In some embodiments, the chemical complex includes the exhaust stream for discharge of oxygen depleted air and combustion products from the retentate side, at least part of the exhaust stream feeding a combustion chamber, at least part of the flue gas from the combustion chamber supplying heat to the oxygen separation module, such that the temperature of the oxygen transport membrane is 850° C. to 1500° C. In some embodiments, the chemical complex includes at least part of the flue gas from the combustion chamber recycled to the oxygen separation module supplying heat, such that the temperature of the oxygen transport membrane is 850° C. to 1500° C.

In some embodiments, the outlet stream for removing oxygen enriched gas and combustion productions from the permeate side, at least part of the outlet stream feeding a combustion chamber, and the exhaust stream for discharge of oxygen depleted air combustion products from the retentate side, at least part of the exhaust stream feeding either the same or a different combustion chamber, at least part of the flue gas or flue gases from the combustion chamber or chambers supplying heat to the oxygen separation module, such that the temperature of the oxygen transport membrane is 850° C. to 1500° C.

In some embodiments, the outlet stream for removing oxygen enriched gas and combustion productions from the permeate side, at least part of the outlet stream feeding a combustion chamber, at least part of the flue gas from the combustion chamber recycled to the oxygen separation module supplying heat, such that the temperature of the oxygen transport membrane is 850° C. to 1500° C.

In some embodiments, the temperature of the oxygen transport membrane is 850° C. to 1250 ° C. In some embodiments, the temperature of the oxygen transport membrane is 850° C. to 1000° C.

In some embodiments, the pressure of the combustion chamber is atmospheric to 700 kPag.

In some embodiments, the chemical complex includes a mixed metal oxide catalyst selected from the group consisting of:

i) catalysts of the formula:

MoₐV_{b}Te_{c}Nb_{d}PdₑO_{f}

wherein a, b, c, d, e and f are the relative atomic amounts of the elements Mo, V, Te, Nb, Pd and O, respectively; and when a=1, b=0.01 to 1.0, c=0 to 1.0, d=0 to 1.0, 0 ≤e≤0.10 and f is a number to satisfy the valence state of the catalyst;

ii) catalysts of the formula:

Ni_{g}AₕBᵢDⱼO_{f}

wherein: g is a number from 0.1 to 0.9, in some cases from 0.3 to 0.9, in other cases from 0.5 to 0.85, and in some situations from 0.6 to 0.8; h is a number from 0.04 to 0.9; i is a number from 0 to 0.5; j is a number from 0 to 0.5; and f is a number to satisfy the valence state of the catalyst; A is selected from the group consisting of Ti, Ta, V, Nb, Hf, W, Y, Zn, Zr, Si and Al or mixtures thereof; B is selected from the group consisting of La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg, and mixtures thereof; D is selected from the group consisting of Ca, K, Mg, Li, Na, Sr, Ba, Cs, and Rb and mixtures thereof; and O is oxygen;

iii) catalysts of the formula:

MoₐEₖGₗO_{f}

wherein: E is selected from the group consisting of Ba, Be, Ca, Cr, Mn, Nb, Ta, Ti, Te, V, W and mixtures thereof; G is selected from the group consisting of Al, Bi, Ce, Co, Cu, Fe, K, Mg, V, Ni, P, Pb, Sb, Si, Sn, Ti, U, and mixtures thereof; a=1; k is 0 to 2; l =0 to 2, with the proviso that the total value of 1 for Co, Ni, Fe and mixtures thereof is less than 0.5; and f is a number to satisfy the valence state of the catalyst;

iv) catalysts of the formula:

VₘMoₙNbₒTeₚMe_{q}O_{f}

wherein: Me is a metal selected from the group consisting of Ta, Ti, W, Hf, Zr, Sb and mixtures thereof; m is from 0.1 to 3; n is from 0.5 to 1.5; o is from 0.001 to 3; p is from 0.001 to 5; q is from 0 to 2; and f is a number to satisfy the valence state of the catalyst;

v) catalysts of the formula:

MoₐVᵣXₛYₜZᵤMᵥO_{f}

wherein: X is at least one of Nb and Ta; Y is at least one of Sb and Ni; Z is at least one of Te, Ga, Pd, W, Bi and Al; M is at least one of Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t =0.001 to 1.0; u=0.001 to 0.5; v=0.001 to 0.3; and f is a number to satisfy the valence state of the catalyst;

vi) a mixed metal oxide having the empirical formula:

Mo_{6.5-7.0}V₃O_{d}

wherein d is a number to at least satisfy the valence of the metals in the catalyst; and

vii) a mixed metal oxide having the empirical formula:

Mo_{6.25-7.25}V₃O_{d}

wherein d is a number to at least satisfy the valence of the metals in the catalyst.

In some embodiments, the chemical complex includes a mixed metal oxide catalyst selected from the group consisting of the formula:

Mo₁V_{0.1-1}Nb_{0.1-1}Te_{0.01-0.2}X_{0-0.2}O_{f}

wherein X is selected from Pd, Sb Ba, Al, W, Ga, Bi, Sn, Cu, Ti, Fe, Co, Ni, Cr, Zr, Ca and oxides and mixtures thereof, and f is a number to satisfy the valence state of the catalyst.

### BRIEF DESCRIPTION OF THE DRAWINGS

To easily identify the discussion of any particular element or act, the most significant digit or digits in a reference number refer to the figure number in which that element is first introduced.

FIG. 1 illustrates a chemical complex of the present disclosure 100 in accordance with one embodiment.

FIG. 2 illustrates a chemical complex of the present disclosure 200 in accordance with one embodiment.

FIG. 3 illustrates a Flow Diagram in accordance with one embodiment.

FIG. 4 illustrates a Flow Diagram in accordance with one embodiment.

### DESCRIPTION OF EMBODIMENTS

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the properties that the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

### Definitions

$$Conversion ( \% ) = \frac{\frac{\begin{matrix}Net mass flow rate of converted {C}_{2} {H}_{6} \\ ( g {C}_{2} {H}_{6} / min )\end{matrix}}{\begin{matrix}Molecular weight of {C}_{2} {H}_{6} \\ ( g {C}_{2} {H}_{6} / mol {C}_{2} {H}_{6} )\end{matrix}}}{\frac{\begin{matrix}Mass flow rate of feed {C}_{2} {H}_{6} \\ ( g {C}_{2} {H}_{6} / min )\end{matrix}}{\begin{matrix}Molecular weight of {C}_{2} {H}_{6} \\ ( g {C}_{2} {H}_{6} / mol {C}_{2} {H}_{6} )\end{matrix}}}$$

“C2-C4 alkane” refers to one of ethane, propane, n-butane or isobutane, or any combination thereof.

“C2-C4 alkene” refers to one of ethylene, propylene, α-butylene, cis-β-butylene, trans-β-butylene, isobutylene, or any combination thereof.

“Conversion” refers to the percentage of C2-C4 alkane carbon atoms fed to the ODH reactor that are converted to carbonaceous products, and can be calculated according to the formula (for ethane):

where the net mass flow of converted C₂H₆ refers and is equal to the mass flow rate of C₂H₆ in the product stream minus the mass flow rate of C₂H₆ in the feed stream.

“Selectivity” refers to the percentage of C2-C4 alkane carbon atoms that are converted to a specific product X in the oxidative dehydrogenation process. For example, in an ethane ODH process, a selectivity of 50% for ethylene indicates 50% of the ethane carbon atoms that are converted during the ODH process are converted into ethylene. Selectivity, is calculated according to the formula:

$$Selectivity ( \% ) = \frac{\frac{\begin{matrix}Net mass flow of X \\ ( g X / min )\end{matrix}}{\begin{matrix}Molecular weight of X \\ ( g X / mol X )\end{matrix}}}{\begin{matrix}[ \frac{\begin{matrix}Net mass flow rate of converted {C}_{2} {H}_{6} \\ ( g {C}_{2} {H}_{6} / min )\end{matrix}}{\begin{matrix}Molecular weight of {C}_{2} {H}_{6} \\ ( g {C}_{2} {H}_{6} / mol {C}_{2} {H}_{6} )\end{matrix}} ] * \\ \frac{Mol . Equiv . of X}{mol {C}_{2} {H}_{6}}\end{matrix}}$$

where X is the product that is being assessed, the net mass flow rate refers to flow in g/min for X or converted C₂H₆ and is equal to the mass flow rate of X or converted C₂H₆ in the product stream minus the mass flow rate of component X or converted C₂H₆ in the feed stream, and molar equivalent (Mol. Equiv.) refers to the amount of X, in moles, that reacts completely with or is produced by one mole of ethane. If the sum of all selectivities for products derived from conversion of ethane did not total 100%, the selectivities were normalized to 100%. Normalization for each product can be calculated by dividing the selectivity for that product by the sum of all carbon atom product selectivities. “Feed stream” refers to a feed stream to an oxidative dehydrogenation reactor, which includes not less than about 20 vol % of C2-C4 alkane, up to about 30 vol % oxygen, and the balance a heat removal diluent gas including N₂, CO₂, Ar, steam or other heat removal diluent gas.

“Flammability envelope” refers to the envelope defining the flammability zone in mixtures of fuel and oxygen, with or without a heat removal diluent gas.

“Gas hourly space velocity” (abbreviated GHSV) refers to the ratio of the gas volumetric flow rate where the gas includes the reacting gas species and optionally one or more heat removal diluent gases at standard temperature and pressure (STP, i.e., 0° C., 1 bar) to the volume of the catalyst bed. The catalyst bed can refer to either the catalyst active phase, or to the total catalyst formulation which can include such things as catalyst additives or promoters.

“Group 4 element” refers to an element from group 4 of the periodic table; the group includes titanium, zirconium and hafnium.

“Group 5 element” refers to an element from group 5 of the periodic table; the group includes vanadium, niobium and tantalum.

“Heat dissipative particles” refers to solid particles that can be added and mixed with a catalyst bed; the heat dissipative particles can dissipate heat from the catalyst bed.

“Heat removal diluent gas” refers to a gas that dilutes a stream and can remove heat from the stream.

“Mixed metal oxide catalyst” refers to a catalyst that can be used in an oxidative dehydrogenation reactor to oxidatively dehydrogenate a C2-C4 alkane to a C2-C4 alkene.

“Residence time” refers to a measure of how much time material that is flowing through a volume spends in the volume. The residence times indicated herein are equal to the volumetric flow rate of the feed stream at standard conditions (i.e., 0° C., 1 bar) divided by volume of the reactor, which is occupied by the catalyst bed in the reactor. Direct correlation of the measured residence times under operating conditions to residence time under standard conditions falls within the knowledge of the person skilled in the art.

“Weight hourly space velocity” (abbreviated WHSV) refers to the ratio of the gas mass flow rate where the gas includes the reacting gas species and optionally one or more heat removal diluent gases to the mass of the catalyst bed. The catalyst bed can refer to either the catalyst active phase, or to the total catalyst formulation which can include such things as catalyst additives or promoters.

Generally, the concept of ODH has been known since at least the 1950s, for example U.S. Pat. No. 3,049,574. Some effort has been made on improving the process, including improving catalyst efficiency and selectivity. In some instances. this has resulted in disclosure of various catalyst types including carbon molecular sieves, metal phosphates, and mixed metal oxides.

In many cases, oxidative dehydrogenation of alkanes includes contacting a mixture of an alkane or alkanes and oxygen in an ODH reactor with an ODH catalyst under conditions that promote oxidation of alkanes into alkenes. Conditions within the reactor are controlled by the operator and can include, but are not limited to, parameters such as temperature, pressure, and flow rate. Conditions often vary and can be optimized for a specific catalyst, or whether a heat removal diluent gas is used in the mixing of the reactants. In some cases, oxidative dehydrogenation can be used to convert alkanes to alkenes, in particular ethane to ethylene. In some instances, certain modules can be used in a chemical complex to perform the oxidative dehydrogenation of ethane to ethylene followed by downstream processing where the target product ethylene is separated, to the extent possible, from by-products, diluent, and unreacted ethane.

In broad terms, the present disclosure relates to a chemical complex for ODH that includes an oxygen separation module with energy supplied by hot feed gas, the energy is provided by combustion in a device external to the oxygen transport membrane.

Another disclosure to an oxidative dehydrogenation chemical complex is provided in U.S. Pat. No. 10,343,957 assigned to NOVA Chemicals (International) S.A., which discloses an oxidative dehydrogenation chemical complex including integration of an oxygen separation module. Disclosed herein is a chemical complex configuration that further includes a process design to avoid unintentional hot spots which could destroy the membrane, and any upsets within the membrane which could lead to temporary extinguishing of a flame and lead to internal explosion and potentially damaging the membrane. The membrane unit would have to be designed to provide deflagration containment design requirements.

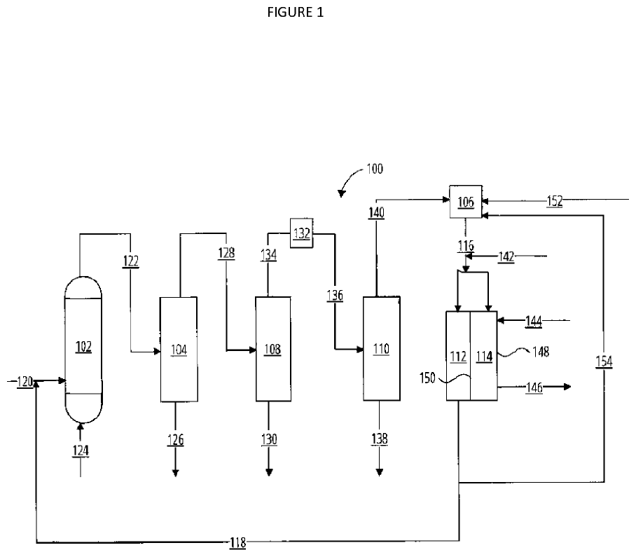

An embodiment of the chemical complex of the present disclosure, shown schematically in FIG. 1, includes, in cooperative arrangement, an ODH Reactor 102, a Quench Tower 104, an Amine Wash Tower 108, a Drier 132, a Distillation Tower 110, a Combustion Chamber 106 and an Oxygen Separation Module 148. ODH Reactor 102 includes an ODH catalyst capable of catalyzing the oxidative dehydrogenation of lower alkane, introduced via Alkane port 124, in the presence of oxygen which may be introduced via Oxygen port 120. The ODH reaction may also occur in the presence of a heat removal diluent gas, such as carbon dioxide, nitrogen, or steam, that is added to ensure the mixture of oxygen and hydrocarbon are outside of the flammability envelope. Determination of whether a mixture is outside of the flammability envelope, for the prescribed temperature and pressure, is within the knowledge of the skilled worker. An ODH reaction that occurs within ODH Reactor 102 may also produce, depending on the catalyst and the prevailing conditions within ODH Reactor 102, a variety of other products which may include carbon dioxide, carbon monoxide, oxygenates, and water. These products leave ODH Reactor 102, along with unreacted alkane, corresponding alkene, residual oxygen, and heat removal diluent gas, if added, via ODH Reactor Product Line 122.

ODH Reactor Product Line 122 is directed to Quench Tower 104 which quenches the products from ODH Reactor Product Line 122 and facilitates removal of oxygenates and water via Quench Tower Bottom Outlet 126. Unconverted lower alkane, corresponding alkene, unreacted oxygen, carbon dioxide, carbon monoxide, and heat removal diluent gas added to Quench Tower 104 exit through Quench Tower Overhead 128 and are directed into Amine Wash Tower 108. Carbon dioxide present in Quench Tower Overhead 128 is isolated by Amine Wash Tower 108, and captured via Carbon Dioxide Bottom Outlet 130 and may be sold, or, alternatively, may be recycled back to ODH Reactor 102 as heat removal diluent gas (not shown). There may be other process steps not shown such as membranes, absorbents, caustic tower, and so on to remove any other contaminants by any means known in the art. Products introduced into Amine Wash Tower 108 via Quench Tower Overhead 128, other than carbon dioxide, leave Amine Wash Tower 108 through Amine Wash Tower Overhead 134 and are passed through a Drier 132 before being directed via Dried Stream Line 136 to Distillation Tower 110, where C2/C2+ hydrocarbons are isolated and removed via C2/C2+ Hydrocarbons Bottom Outlet 138. The remainder includes mainly C1 hydrocarbons, remaining heat removal diluent gas and carbon monoxide, which leave Distillation Tower 110 via Overhead Stream 140 that is directed to a Combustion Chamber 106. Overhead Stream 140 is at least partially combusted in the Combustion Chamber 106. The exit from Combustion Chamber 106 is Flue Gas Line 116.

Combustion Chamber 106 can be used to provide energy to the Oxygen Separation Module 148. The energy can be supplied by combusting fuel from Fuel Line 152 and Overhead Stream 140 in Combustion Chamber 106, the hot effluent enters Flue Gas Line 116 the energy to either the Permeate Side 112 or Retentate Side 114 of Oxygen Separation Module 148.

Oxygen Separation Module 148 includes a sealed vessel having a Retentate Side 114 and a Permeate Side 112, separated by Oxygen Transport Membrane 150. Optionally, a flow of feed air via Feed Air Line 142 is mixed with the contents of Flue Gas Line 116 prior to entering the Oxygen Separation Module 148. Optionally the contents of Flue Gas Line 116 enter the Permeate Side 112, and feed air from Feed Air Line 142 enters the Retentate Side 114. Optionally, a flow controlling means (not shown) may be included that allows for flow into both sides at varying levels. In that instance, an operator may choose what portion of the flow from Flue Gas Line 116 enters Retentate Side 114 and what portion enters Permeate Side 112. Depending upon conditions, an operator may switch between the two sides, allow equivalent amounts to enter each side, or bias the amount directed to one of the two sides. Optionally, the feed air is fed separately to two flows from Flue Gas Line 116 which can enter both the Retentate Side 114 and the Permeate Side 112 and can be fed at different levels and enter both the Retentate Side 114 and the Permeate Side 112 in varying concentrations. Oxygen Separation Module 148 also includes Air Input 144 for the introduction of atmospheric air, or other oxygen containing gas, into the Retentate Side 114. Combustion takes place in Combustion Chamber 106, the products of which can be introduced into Retentate Side 114 and may contribute to raising or maintaining the temperature of Oxygen Transport Membrane 150 to at least about 850° C. so that oxygen can pass from Retentate Side 114 to Permeate Side 112. Components within the atmospheric air, or other oxygen containing gas, other than oxygen, cannot pass from Retentate Side 114 to Permeate Side 112 and can only leave Oxygen Separation Module 148 via O₂ Depleted Air Exhaust Line 146.

As a result of oxygen passing from Retentate Side 114 to Permeate Side 112, there is separation of oxygen from atmospheric air, or other oxygen containing gas, introduced into Retentate Side 114. The result is production of oxygen enriched gas on Permeate Side 112, which is then directed via oxygen O₂ Enriched Permeate Line 118 to ODH Reactor 102, either directly or in combination with Oxygen port 120 (as shown in FIG. 1). When the contents of Flue Gas Line 116 are directed into Retentate Side 114 the degree of purity of oxygen in O₂ Enriched Permeate Line 118 can approach 99%. Conversely, when the contents of Flue Gas Line 116 are directed into Permeate Side 112, the degree of purity of oxygen in O₂ Enriched Permeate Line 118 is lower, with an upper limit ranging from about 80 to about 90% oxygen; the balance in the form of carbon dioxide, water, and remaining heat removal diluent gas, all of which do not affect the ODH reaction as contemplated by the present disclosure and can accompany the oxygen in O₂ Enriched Permeate Line 118 into ODH Reactor 102. Water and carbon dioxide are ultimately removed by Quench Tower 104 and Amine Wash Tower 108, respectively. Indeed, one of the advantages of this disclosure is that carbon dioxide can be captured for sale as opposed to being flared where it contributes to greenhouse gas emissions. Alternatively, when carbon dioxide is used as the heat removal diluent gas, any carbon dioxide captured in the amine wash can be recycled back to ODH Reactor 102 to perform its role as heat removal diluent gas and/or as a reactant.

Oxygen Transport Membrane 150 is temperature dependent, only allowing transport of oxygen when the temperature reaches at least about 850° C.

The temperature of the contents within ODH Reactor Product Line 122, in a typical ODH process can reach about 450° C. It may be desirable to lower the temperature of the ODH Reactor Product Line 122 before introduction into Quench Tower 104. In that instance, the present disclosure contemplates the use of a heat exchanger immediately downstream of each ODH Reactor 102 and immediately upstream of the Quench Tower 104. Use of a heat exchanger to lower temperatures in this fashion is well known in the art.

The pressures within the Oxygen Separation Module should be controlled, such that the partial pressure of oxygen on the Permeate Side is lower than the oxygen partial pressure on the Retentate Side. This ensures that oxygen has a driving force which moves the oxygen from the Retentate Side to the Permeate Side of the Oxygen Transport Membrane. The partial pressures of oxygen can be monitored and controlled by any means known to a person of ordinary skill in the art.

In an embodiment of the present disclosure, a stream in O₂ Burn Line 154 can be split from the stream in O₂ Enriched Permeate Line 118 and can be fed to Combustion Chamber 106. Combustion Chamber 106 can also receive a stream of fuel from Fuel Line 152. The Combustion Chamber 106 can have at least one outlet Flue Gas Line 116, the contents of which can be recycled and can form part of the feed to the Oxygen Separation Module 148.

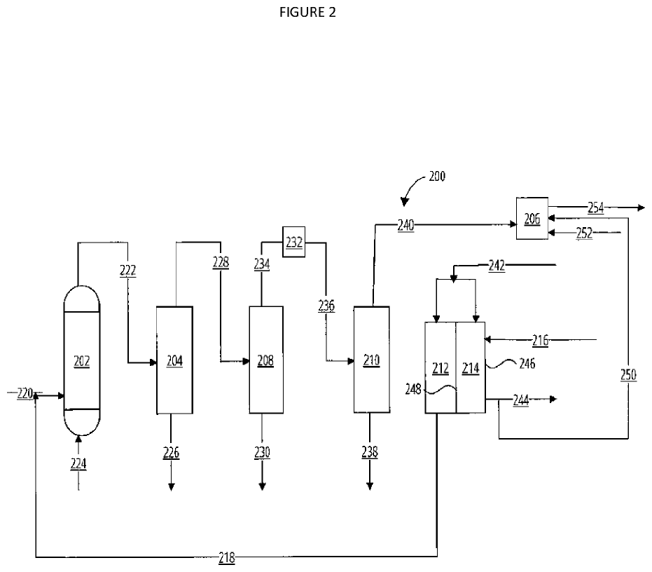

The chemical complex of the present disclosure, shown in one embodiment schematically in FIG. 2, includes, in cooperative arrangement, an ODH Reactor 202, a Quench Tower 204, an Amine Wash Tower 208, a Drier 232, a Distillation Tower 210, and an Oxygen Separation Module 246. ODH Reactor 202 includes an ODH catalyst capable of catalyzing the oxidative dehydrogenation of lower alkane, introduced via Alkane port 224, in the presence of oxygen which may be introduced via Oxygen port 220. The ODH reaction may also occur in the presence of a heat removal diluent gas, such as carbon dioxide, nitrogen, or steam, that is added to ensure the mixture of oxygen and hydrocarbon are outside of the flammability envelope. Determination of whether a mixture is outside of the flammability envelope, for the prescribed temperature and pressure, is within the knowledge of the skilled worker. An ODH reaction that occurs within ODH Reactor 202 may also produce, depending on the catalyst and the prevailing conditions within ODH Reactor 202, a variety of other products which may include carbon dioxide, carbon monoxide, oxygenates, and water. These products leave ODH Reactor 202, along with unreacted alkane, corresponding alkene, residual oxygen, and heat removal diluent gas, if added, via ODH Reactor Product Line 222.

ODH Reactor Product Line 222 is directed to Quench Tower 204 which quenches the products from ODH Reactor Product Line 222 and facilitates removal of oxygenates and water via Quench Tower Bottom Outlet 226. Unconverted lower alkane, corresponding alkene, unreacted oxygen, carbon dioxide, carbon monoxide, and heat removal diluent gas added to Quench Tower 204 exit through Quench Tower Overhead 228 and are directed into Amine Wash Tower 208.

Carbon dioxide present in Quench Tower Overhead 228 is isolated by Amine Wash Tower 208 and captured via Carbon Dioxide Bottom Outlet 230 and may be sold, or, alternatively, may be recycled back to ODH Reactor 202 as heat removal diluent gas (not shown). There may be other process steps not shown such as membranes, absorbents, caustic tower, and so on to remove any other contaminants by any means known in the art. Products introduced into Amine Wash Tower 208 via Quench Tower Overhead 228, other than carbon dioxide, leave Amine Wash Tower 208 through

Amine Wash Tower Overhead 234 and are passed through a Drier 232 before being directed via Dried Stream Line 236 to Distillation Tower 210, where C2/C2+ hydrocarbons are isolated and removed via C2/C2+ Hydrocarbons Bottom Outlet 238. The remainder includes mainly C1 hydrocarbons, remaining heat removal diluent gas and carbon monoxide, which leave Distillation Tower 210 via Overhead Stream 240 that is directed to Combustion Chamber 206. Overhead Stream 240 is at least partially combusted in the Combustion Chamber 206. The exit of the Combustion Chamber 206 is Flue Gas Line 254.

Oxygen Separation Module 246 includes a sealed vessel having a Retentate Side 214 and a Permeate Side 212, separated by Oxygen Transport Membrane 248. A stream of feed air from Feed Air Line 242 may be directed into either of Retentate Side 214 or Permeate Side 212. Optionally, a flow controlling means (not shown) may be included that allows for flow into both sides at varying levels. In that instance, an operator may choose what portion of the flow from Feed Air Line 242 enters Retentate Side 214 and what portion enters Permeate Side 212. Depending upon conditions, an operator may switch between the two sides, allow equivalent amounts to enter each side, or bias the amount directed to one of the two sides. Oxygen Separation Module 246 can also include a stream for the introduction of atmospheric air, or other oxygen containing gas, into the Retentate Side 214.

Components within the atmospheric air, or other oxygen containing gas, other than oxygen, cannot pass from Retentate Side 214 to Permeate Side 212 and can only leave Oxygen Separation Module 246 via O₂ Depleted Air Exhaust Line 244.

As a result of oxygen passing from Retentate Side 214 to Permeate Side 212, there is separation of oxygen from atmospheric air, or other oxygen containing gas, introduced into Retentate Side 214. The result is production of oxygen enriched gas on Permeate Side 212, which is then directed via oxygen in O₂ Enriched Permeate Line 218 to ODH Reactor 202, either directly or in combination with Oxygen port 220 (as shown in FIG. 2). When Feed Air Line 242 is directed into Retentate Side 214 the degree of purity of oxygen in O₂ Enriched Permeate Line 218 can approach 99%. Conversely, when feed air from Feed Air Line 242 is directed into Permeate Side 212, the degree of purity of oxygen in O₂ Enriched Permeate Line 218 is lower, with an upper limit ranging from about 80 to about 90% oxygen; the balance in the form of carbon dioxide, water, and remaining heat removal diluent gas, all of which do not affect the ODH reaction as contemplated by the present disclosure and can accompany the contents in O₂ Enriched Permeate Line 218 into ODH Reactor 202. Water and carbon dioxide are ultimately removed by Quench Tower 204 and Amine Wash Tower 208, respectively. Indeed, one of the advantages of this disclosure is that carbon dioxide can be captured for sale as opposed to being flared where it contributes to greenhouse gas emissions. Alternatively, when carbon dioxide is used as the heat removal diluent gas, any carbon dioxide captured in the amine wash can be recycled back to ODH Reactor 202 to perform its role as heat removal diluent gas.

Oxygen Transport Membrane 248 is temperature dependent, only allowing transport of oxygen when the temperature reaches at least 850° C. In some instances, the components in Feed Air Line 242 by themselves are not capable, upon combustion in the presence of oxygen, to raise the temperature of Oxygen Transport Membrane 248 to the required level. For this reason, the chemical complex described in this disclosure also includes heat transfer from the stream contained in Flue Gas Line 254 to Feed Air Line 242, upstream of Oxygen Separation Module 246.

The temperature of the contents within ODH Reactor Product Line 222, in a typical ODH process can reach about 450° C. It may be desirable to lower the temperature of the ODH Reactor Product Line 222 before introduction into Quench Tower 204. In that instance, the present disclosure contemplates the use of a heat exchanger immediately downstream of each ODH Reactor 202 and immediately upstream of the Quench Tower 204. Use of a heat exchanger to lower temperatures in this fashion is well known in the art.

The pressures within the Oxygen Separation Module should be controlled, such that the partial pressure of oxygen on the Permeate Side is lower than the oxygen partial pressure on the Retentate Side. This ensures that oxygen has a driving force which moves the oxygen from the Retentate Side to the Permeate Side of the Oxygen Transport Membrane. The partial pressures of oxygen can be monitored and controlled by any means known to a person of ordinary skill in the art.

In an embodiment of the present disclosure, a stream in O₂ Burn Line 250 can be split from the stream in O₂ Depleted Air Exhaust Line 244 and can be fed to Combustion Chamber 206. Combustion Chamber 206 can also receive a stream of fuel from Fuel Line 252. The Combustion Chamber 206 can have at least one outlet Flue Gas Line 254, the contents of which can be recycled and can form part of the feed to the Retentate Side 214 of the Oxygen Separation Module 246. In an embodiment of the present disclosure, the contents in O₂ Depleted Air Exhaust Line 244 can be used to preheat the contents in Feed Air Line 242. In an embodiment of the present disclosure, the contents of O₂ Depleted Air Exhaust Line 244 and the contents of Flue Gas 254 Line can be used to preheat the contents of Feed Air Line 242. In an embodiment of the present disclosure, the contents of Flue Gas Line 254 can be used to preheat the contents in Feed Air Line 242. The feed air in Feed Air Line 242 can be heated in Combustion Chamber 206 indirectly by a fired heater. O₂ Enriched Permeate Line 218 will need to be cooled down to below about 250° C. before its contents can be sent to ODH Reactor 202, so in an embodiment of the present disclosure, O₂ Enriched Permeate Line 218 can be cooled by the contents in Feed Air Line 242. In an embodiment of the present disclosure, one, some, or all of the methods mentioned above can be used in combination to heat the contents of Feed Air Line 242.

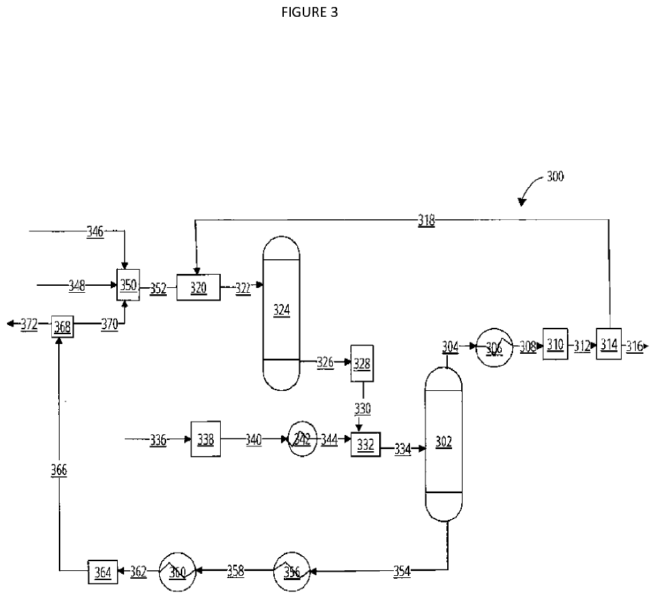

The Flow Diagram 300 shown in one embodiment schematically in FIG. 3 (and used in an AspenPlus® Software Model, Aspen Technology, Inc., in Example 2) includes an Oxygen Separation Module 302, including two outlet streams, one in Permeate High Temperature Line 304, and one in Retentate High Temperature Line 354. The stream in Stream Permeate High Temperature Line 304 can enter a cooler Heat Exchanger 306, which includes an outlet stream in Permeate Low Temperature Line 308, which can experience a pressure drop in a Valve 310. An outlet of Valve 310 provides a stream in Permeate Line 312, which can be split using Permeate Splitter 314, providing two outlet streams, one in Permeate Purge Line 316 and one in Permeate Recycle Line 318. The stream in Permeate Recycle Line 318 can mix with the stream in Mixed Gas Line 352 in Mixer 320, an outlet of Mixer 320 can be provided to Combustion Chamber Feed Line 322, which can enter Combustion Chamber 324. An outlet of Combustion Chamber 324 includes a Flue Gas Low Pressure Line 326, the contents of which can be compressed by Compressor 328 to become a stream in Flue Gas High Pressure Line 330. The stream in Flue Gas High Pressure Line 330 can be mixed with an air stream Air High Temperature 344 in a Mixer 332. Mixer 332 can have an outlet feed to Membrane 334 which can enter Oxygen Separation Module 302. A stream included in Air Line 336 can enter Compressor 338, an outlet of Compressor 338, can be provided to Air High Pressure Line 340, can then be heated by Heat Exchanger 342, the stream then provided via Air High Temperature Line 344. A stream in CO Line 346 can be mixed with fuel from Fuel Line 348 in a Mixer 350, the mixed gas can be transported via Mixed Gas Line 352 which can be mixed with a stream in Permeate Recycle Line 318 in Mixer 320. A retentate stream from Oxygen Separation Module 302, a stream from Retentate High Temperature Line 354, can be cooled in Heat Exchanger 356 becoming a stream in Retentate Mid Temperature Line 358, which can be cooled again by Heat Exchanger 360, which can become a stream in Retentate Low Temperature Line 362. The stream in Retentate Low Temperature Line 362 can experience a pressure drop using a Valve 364, becoming a stream in Retentate Low Pressure Line 366. The stream in Retentate Low Pressure Line 366 can be split in a Splitter 368, to provide an outlet stream from Splitter 368, which can be recycled to Mixer 350 as a stream in Retentate Recycle Line 370, and another outlet stream can be provided to Retentate Purge Line 372.

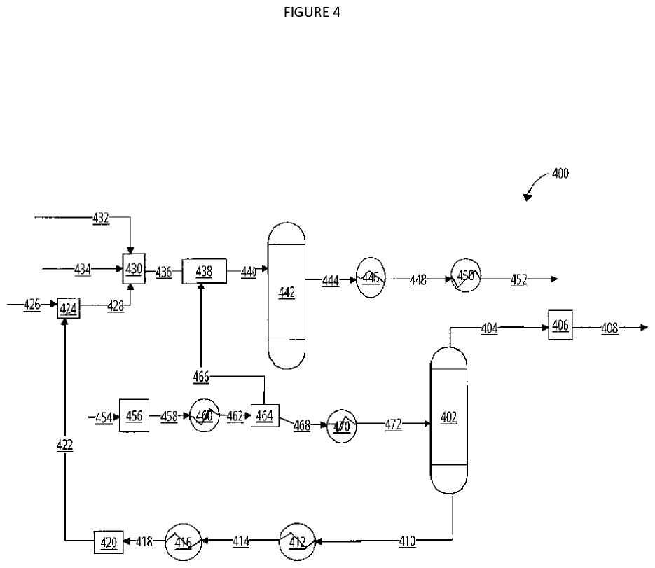

The Flow Diagram 400, shown in one embodiment schematically in FIG. 4 (and used in Example 3 with AspenPlus® Software Model, Aspen Technology, Inc.) includes an Oxygen Separation Module 402, including two outlet streams, Permeate High Pressure Line 404, and Retentate High Temperature Line 410. Stream Permeate High Pressure Line 404 can experience a pressure drop using a Valve 406, which can form a stream in Permeate Line 408. A stream from Oxygen Separation Module 402, a stream Retentate High Temperature Line 410, can be cooled in Cooler 412. An outlet of this cooler, the stream in Retentate Mid Temperature Line 414, can be further cooled using Cooler 416. An outlet of Cooler 416, a stream in Retentate Low Temperature Line 418, can have a pressure drop via Valve 420, becoming a stream in Retentate Line 422. The stream in Retentate Line 422 can be mixed in Mixer 424, with a stream of nitrogen and CO₂ in Line 426. An outlet of Mixer 424, a stream in Retentate Dilute Line 428, can be mixed in Mixer 430 with a CO stream in Line 432, and a furl stream from Fuel Line 434. A resulting stream of fuel and retentate from Fuel and Retentate Line 436, can enter a Mixer 438, becoming a stream in Combustion Chamber Feed Line 440, which can enter Combustion Chamber 442. An outlet of the Combustion Chamber 442 provides a stream to Flue Gas High Temperature Line 444, the stream then can enter Heat Exchanger 446, the stream then enters Flue Gas Mid Temperature Line 448. The stream in Flue Gas Mid Temperature Line 448 can enter Heat Exchanger 450, becoming a stream in Flue Gas Line 452. A stream of air in Air Line 454 can enter Compressor 456, becoming a stream in Air High Pressure Line 458, which can enter Heat Exchanger 460, becoming a stream in Air High Temperature Line 462. The stream in Air High Temperature Line 462 can enter Splitter 464, and be split into streams included in Air to Combustion Chamber Line 466 which can connect to Mixer 438, and a stream in Air to Membrane Line 468, which can enter Heat Exchanger 470, and become a stream in Feed to Membrane Line 472 which can enter Oxygen Separation Module 402.

### ODH Process

ODH of C2-C4 alkanes includes contacting a mixture of a C2-C4 alkane and oxygen in one or more ODH reactors with one or more mixed metal oxide catalysts under conditions that promote oxidation of the C2-C4 alkane into its corresponding C2-C4 alkene. Conditions within the reactor are controlled by the operator and include, but are not limited to, parameters such as temperature, pressure, and flow rate. Conditions will vary and can be optimized for a particular C2-C4 alkane, or for a specific mixed metal oxide catalyst, or whether a heat removal diluent gas or heat dissipative particles are used in the mixing of the reactants.

In embodiments of the disclosure, the C2-C4 alkane comprises ethane, and its corresponding C2-C4 alkene comprises ethylene.

Any of the known reactor types applicable for the ODH of alkanes may be used with the methods disclosed herein. The methods may be used with conventional fixed bed reactors, fluidized bed reactors, ebulliated bed reactors, rotating bed reactors, swing bed reactors, etc. In a typical fixed bed reactor, reactants are introduced into the reactor at one end, flow past an immobilized catalyst, products are formed and leave at the other end of the reactor. Designing a fixed bed reactor suitable for the methods disclosed herein can follow techniques known for reactors of this type. A person skilled in the art would know which features are required with respect to shape and dimensions, inputs for reactants, outputs for products, temperature and pressure control, and means for immobilizing the catalyst.

The methods may be used with conventional fluidized bed reactors, where the catalyst bed can be supported by a porous structure, or a distributor plate, located near a bottom end of the reactor and reactants flow through at a velocity sufficient to fluidize the bed. The reactants are converted to products upon contact with the fluidized catalyst and the reactants are subsequently removed from the upper end of the reactor. A fluidized bed could also be used in a process in which the catalyst is regenerated in a regeneration bed and then returned to the fluidized bed. Design considerations those skilled in the art can modify and optimize include, but are not limited to, the shape of the reactor, the shape and size of the distributor plate, the input temperature, the output temperature, and reactor temperature and pressure control.

Embodiments of the disclosure include using a combination of both fixed bed and fluidized bed reactors, each with the same or different ODH mixed metal oxide catalyst. The multiple reactors can be arrayed in series or in parallel configuration, the design of which falls within the knowledge of the worker skilled in the art.

Use of an ODH reactor for performing an ODH process consistent with the present disclosure falls within the knowledge of the person skilled in the art. For best results, the ODH of a C2-C4 alkane may be conducted at temperatures from about 300° C. to about 500° C., typically from about 300° C. to about 425° C., often from about 330° C. to about 400° C., at pressures from about 0.5 to about 100 psig (3.447 to 689.47 kPag), often from about 15 to about 50 psig (103.4 to 344.73 kPag), and the residence time is typically from about 0.10 to about 10 seconds, often from about 1 to about 5 seconds.

In some instances, the ODH process has a selectivity for the corresponding C2-C4 alkene (ethylene in the case of ethane ODH) of greater than about 85%, often greater than about 90%. The flow of reactants and heat removal diluent gas can be described in any number of ways known in the art. Typically, flow is described and measured in relation to the volume of all feed gases (reactants and diluent) that pass over the volume of the active catalyst bed in one hour, or gas hourly space velocity (GHSV). The GHSV can range from about 500 to about 30000 h⁻¹, often greater than about 1000 h⁻¹. The flow rate can also be measured as weight hourly space velocity (WHSV), which describes the flow in terms of the weight, as opposed to volume, of the gases that flow over the weight of the active catalyst per hour. In calculating WHSV the weight of the gases may include only the reactants but may also include heat removal diluent gas added to the gas mixture. When including the weight of diluents, when used, the WHSV may range from about 0.5 h⁻¹ to about 50 h⁻¹, often from about 1.0 to about 25.0 h⁻¹.

The flow of gases through the ODH reactor may also be described as the linear velocity of the gas stream (cm/s), which is defined in the art as the flow rate of the gas stream divided by the cross-sectional surface area of the reactor all divided by the void fraction of the mixed metal oxide catalyst bed. The flow rate generally means the total of the volumetric flow rates of all the gases entering the reactor and is measured where the oxygen and C2-C4 alkane first contact the mixed metal oxide catalyst and at the temperature and pressure at that point. The cross-section of the ODH reactor is also measured at the entrance of the mixed metal oxide catalyst bed. The void fraction of the mixed metal oxide catalyst bed is defined as the volume of voids in the catalyst bed/total volume of the catalyst bed. The volume of voids refers to the voids between catalyst particles and does not include the volume of pores inside the catalyst particles. The linear velocity can range from about 0.5 cm/sec to about 3000 cm/sec, often from about 5 cm/sec to about 1500 cm/sec, often from about 10 cm/sec to about 500 cm/sec.

The space-time yield of corresponding C2-C4 alkene (productivity) in g/hour per kg of the mixed metal oxide catalyst should be not less than about 100, often, greater than about 1500, most often, greater than about 3000, in many cases, greater than about 3500 at about 350° C. to about 400° C. It should be noted that the productivity of the mixed metal oxide catalyst will increase with increasing temperature until the selectivity is decreased.

The use of non-catalytic heat dissipative particles can be used within one or more of the ODH reactors. The heat dissipative particles can be present within the mixed metal oxide catalyst bed and include one or more non-catalytic inert particulates having a melting point at least about 30° C., in some embodiments at least about 250° C., in further embodiments at least about 500° C. above the temperature upper control limit for the reaction; a particle size in range of about 0.5 to about 75 mm, in some embodiments about 0.5 to about 15 mm, in further embodiments in range of about 0.5 to about 8 mm, in further embodiments in the range of about 0.5 to about 5 mm; and a thermal conductivity of greater than about 30 W/mK (watts/meter Kelvin) within the reaction temperature control limits. In some embodiments the particulates are metals and/or metal alloys and compounds having a thermal conductivity of greater than about 50 W/mK (watts/meter Kelvin) within the reaction temperature control limits. Non-limiting examples of suitable metals that can be used in these embodiments include, but are not limited to, silver, copper, gold, aluminum, steel, stainless steel, molybdenum, and tungsten. The heat dissipative particles can have a particle size of from about 1 mm to about 15 mm. In some embodiments, the particle size can be from about 1 mm to about 8 mm. The heat dissipative particles can be added to the bed in an amount from about 5 to about 95 wt. %, in some embodiments from about 30 to about 70 wt. %, in other embodiments from about 45 to about 60 wt. % based on the entire weight of the bed. The particles are employed to potentially improve cooling homogeneity and reduction of hot spots in the bed by transferring heat directly to the walls of the reactor. The heat dissipative particles can optionally be pressed or extruded with the mixed metal oxide catalyst active phase.

### ODH Catalyst

Any of the mixed metal oxide catalysts used as ODH catalysts known in the art are suitable for use in the methods disclosed herein. Non-limiting examples of suitable oxidative dehydrogenation catalyst include those containing one or more mixed metal oxides selected from:

i) catalysts of the formula:

MoₐV_{b}Te_{c}Nb_{d}PdₑO_{f}

wherein a, b, c, d, e and f are the relative atomic amounts of the elements Mo, V, Te, Nb, Pd and O, respectively; and when a=1, b=0.01 to 1.0, c=0 to 1.0, d=0 to 1.0, 0 ≤e≤0.10 and f is a number to at least satisfy the valence state of the metals present in the catalyst;

ii) catalysts of the formula:

Ni_{g}AₕBᵢDⱼO_{f}

wherein g is a number from 0.1 to 0.9, in many cases from 0.3 to 0.9, in other cases from 0.5 to 0.85, in some instances 0.6 to 0.8; h is a number from 0.04 to 0.9; i is a number from 0 to 0.5; j is a number from 0 to 0.5; and f is a number to at least satisfy the valence state of the metals in the catalyst; A is chosen from Ti, Ta, V, Nb, Hf, W, Y, Zn, Zr, Si and Al or mixtures thereof; B is chosen from La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg, and mixtures thereof; D is chosen from Ca, K, Mg, Li, Na, Sr, Ba, Cs, and Rb and mixtures thereof; and 0 is oxygen;

iii) catalysts of the formula:

MoₐEₖGₗO_{f}

wherein E is chosen from Ba, Be, Ca, Cr, Mn, Nb, Ta, Ti, Te, V, W and mixtures thereof; chosen from Al, Bi, Ce, Co, Cu, Fe, K, Mg, V, Ni, P, Pb, Sb, Si, Sn, Ti, U, and mixtures thereof; a=1; k is 0 to 2; l=0 to 2, with the proviso that the total value of 1 for Co, Ni, Fe and mixtures thereof is less than 0.5; and f is a number to at least satisfy the valence state of the metals in the catalyst;

iv) catalysts of the formula:

VₘMoₙNbₒTeₚMe_{q}O_{f}
wherein Me is chosen from Ta, Ti, W, Hf, Zr, Sb and mixtures thereof; m is from 0.1 to 3; n is from 0.5 to 1.5; o is from 0 to 3; p is from 0.001 to 5; q is from 0 to 2; and f is a number to at least satisfy the valence state of the metals in the catalyst;

v) catalysts of the formula:

MoₐVᵣXₛYₜZᵤMᵥO_{f}

wherein X is at least one of Nb and Ta; Y is at least one of Sb and Ni; Z is at least one of Te, Ga, Pd, W, Bi and Al; M is at least one of Be, Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t=0.001 to 1.0; u=0.001 to 0.5; v=0.001 to 0.3; and f is a number to at least satisfy the valence state of the metals in the catalyst;

vi) a mixed metal oxide having the empirical formula:

Mo_{6.5-7.0}V₃O_{d}

wherein d is a number to at least satisfy the valence of the metals in the catalyst; and

vii) a mixed metal oxide having the empirical formula:

Mo_{6.25-7.25}V₃O_{d}

wherein d is a number to at least satisfy the valence of the metals in the catalyst.

An implementation of an ODH catalyst material is a mixed metal oxide having the formula Mo₁V_{0.1-1}Nb_{0.1-1}Te_{0.01-0.2}X_{0-0.2}O_{f} wherein X is selected from Pd, Sb, Ba, Al, W, Ga, Bi, Sn, Cu, Ti, Fe, Co, Ni, Cr, Zr, Ca and oxides and mixtures thereof, and f is a number to satisfy the valence state of the metals present in the catalyst.

An implementation of an ODH catalyst material is a mixed metal oxide that includes Mo, V, O, and iron (Fe). The molar ratio of Mo to V can be from 1:0.25 to 1:0.50 or from 1:0.30 to 1:0.45, or from 1:0.30 to 1:0.35, or from 1:0.35 to 1:0.45. The molar ratio of Mo to Fe can be from 1:0.25 to 1:5.5, or from 1:3 to 1:5.5, or from 1:4.25 to 1:4.75, or from 1:4.45 to 1:4.55, or from 1:0.1 to 1:1, or from 1:0.25 to 1:0.75, or from 1:0.4 to about 1:0.6, or about 1:0.4, or about 1:0.6, or from 1:1.3 to 1:2.2, or from 1:1.6 to 1:2.0, or from 1:1.80 to 1:1.90. Further, oxygen is present at least in an amount to satisfy the valence state of the metals present in the catalyst. The catalyst can have at least a portion of the Fe in the catalyst material present as Fe(III). The catalyst can have at least a portion of the Fe in the catalyst material present as amorphous iron. The catalyst can have at least a portion of the Fe in the catalyst material present as an iron oxide, an iron oxide hydroxide, or a combination thereof. The iron oxide can include an iron oxide selected from hematite (α-Fe₂O₃), maghemite (γ-Fe₂O₃), magnetite (Fe₃O₄), or a combination thereof. The iron oxide hydroxide can include an iron oxide hydroxide selected from a goethite, an akageneite, a lepidocrocite, or a combination thereof. The catalyst can include at least a portion of the iron as a goethite and at least a portion of the iron as a hematite.

An implementation of an ODH catalyst material is a mixed metal oxide having the empirical formula Mo₁V_{0.25-0.5}O_{d} wherein d is a number to satisfy the valence state of the metals present in the catalyst. The molar ratio of Mo to V can be from 1:0.25 to 1:0.5, or 1:0.3 to 1:0.49.

An implementation of an ODH catalyst material is a mixed metal oxide that includes Mo, V, O, and aluminum (Al). The molar ratio of Mo to V can be from 1:0.1 to 1:0.50, or from 1:0.25 to 1:0.50, or from 1:0.3 to 1:0.49, or from 1:0.30 to 1:0.45, or from 1:0.30 to 1:0.35, or from 1:0.35 to about 1:0.45. The molar ratio of Mo to Al is from 1:1.5 to 1:6.5, or from 1:3.0 to 1:6.5, or from 1:3.25 to 1:5.5.5, or from 1:3.5 to 1:4.1, or from 1:4.95 to 1:5.05, or from 1:4.55 to 1:4.65, or from 1:1.5 to 1:3.5, or from 1:2.0 to 1:2.2, or from 1:2.9 to 1:3.1. Oxygen is present at least in an amount to satisfy the valence state of the metals present in the catalyst. At least a portion of the Al in the catalyst material can be present as an aluminum oxide; the aluminum oxide can be an aluminum oxide hydroxide. The aluminum oxide hydroxide can include an aluminum oxide hydroxide selected from a gibbsite, a bayerite, a boehmite, or a combination thereof. At least a portion of the Al in the catalyst material can be present as gamma alumina.

An implementation of an ODH catalyst material is a mixed metal oxide that includes Mo, V, O, Al, and Fe. The molar ratio of Mo to V can be from 1:0.1 to 1:0.5, or from 1:0.30 to 1:0.45, or from 1:0.30 to 1:0.35, or from 1:0.35 to 1:0.45. The molar ratio of Mo to Al can be from 1:1.5 to 1:6.0. The molar ratio of Mo to Fe can be from 1:0.25 to 5:5. Oxygen is present at least in an amount to satisfy the valence state of the metals present in the catalyst. The molar ratio of Mo to Fe can be from 1:0.1 to 1:1, and the molar ratio of Mo to Al can be from 1:3.5 to 1:5.5. The molar ratio of Mo to Fe can be from 1:0.25 to 1:0.75, and the molar ratio of Mo to Al can be from 1:3.75 to 1:5.25. The molar ratio of Mo to Fe can be from 1:0.35 to 1:0.65, and the molar ratio of Mo to Al can be from 1:3.75 to 1:5.25. The molar ratio of Mo to Fe can be from 1:0.35 to 1:0.45, and the molar ratio of Mo to Al can be from 1:3.9 to 1:4.0. The molar ratio of Mo to Fe can be from 1:0.55 to 0:65, and the molar ratio of Mo to Al can be from 1:4.95 to 1:5.05. The molar ratio of Mo to Fe can be from 1:1.3 to 1:2.2, and the molar ratio of Mo to Al can be from 1:2.0 to 1:4.0. The molar ratio of Mo to Fe can be from 1:1.6 to 1:2.0, and the molar ratio of Mo to Al can be from 1:2.5 to 1:3.5. The molar ratio of Mo to Fe can be from 1:1.80 to 1:1.90, and the molar ratio of Mo to Al can be from 1:2.9 to 1:3.1. At least a portion of the Fe in the catalyst material can be present as Fe(III). At least a portion of the Fe in the catalyst material can be present as amorphous Fe. At least a portion of the Fe in the catalyst material can be present as an iron oxide, an iron oxide hydroxide, or a combination thereof. In some embodiments, the iron oxide includes an iron oxide selected from hematite (α-Fe₂O₃), maghemite (γ-Fe₂O), magnetite (Fe₃O₄), or a combination thereof. Iron oxide hydroxide can include an iron oxide hydroxide selected from a goethite, an akageneite, a lepidocrocite, or a combination thereof. At least a portion of the Fe in the catalyst material can be present as a goethite and at least a portion of the Fe in the catalyst material can be present a hematite. At least a portion of the Al in the catalyst material can be is present as an aluminum oxide. The aluminum oxide can include an aluminum oxide hydroxide. The aluminum oxide hydroxide can include an aluminum oxide hydroxide selected from a gibbsite, a bayerite, a boehmite, or a combination thereof. At least a portion of the aluminum in the catalyst material can be present as a gamma alumina.

An implementation of an ODH catalyst material is a mixed metal oxide that includes Mo, V, Be, and O. The molar ratio of Mo to V can be from 1:0.25 to 1:0.65, or from 1:0.35 to 1:0.55, or from 1:0.38 to 1:0.48. The molar ratio of Mo to Be can be from 1:0.25 to 1:0.85, or from 1:0.35 to 1:0.75, or from 1:0.45 to 1:0.65. Oxygen is present at least in an amount to satisfy the valence state of the metals present in the catalyst.

An implementation of an ODH catalyst material is a mixed metal oxide that includes Mo, V, Be, Al and O. The molar ratio of Mo to V can be from 1:0.25 to 1:0.65, or from 1:0.35 to 1:0.55, or from 1:0.38 to 1:0.48. The molar ratio of Mo to Be can be from 1:0.25 to 1:1.7, or from 1:0.35 to 1:0.75, or from 1:0.45 to 1:0.65. The molar ratio of Mo to Al can be from 1:1 to 1:9, or from 1:2 to 1:8, or from 1:4 to 1:6. Oxygen is present at least in an amount to satisfy the valence state of the metals present in the catalyst. At least a portion of the aluminum in the catalyst material can be present as an aluminum oxide. The aluminum oxide can include an aluminum oxide hydroxide. The aluminum oxide hydroxide can include an aluminum oxide hydroxide selected from a gibbsite, a bayerite, a boehmite, or a combination thereof. At least a portion of the aluminum in the catalyst material can be present as gamma alumina.

An implementation of an ODH catalyst material has an amorphous phase of from 20 wt. % to 50 wt. %, or from 25 wt. % to 45 wt. %, or from 45 wt. % to 75 wt. %, or from 55 wt. % to 65 wt. %, or from 50 wt. % to 85 wt. %, or from 55 wt. % to 75 wt. %, or from 60 wt. % to 70 wt. %.

An implementation of an ODH catalyst material has an average crystallite size of greater than about 50 nm, or greater than about 75 nm, or greater than about 100 nm, or greater than about 125 nm, or from about 75 nm to about 150 nm, or from about 75 nm to about 250 nm, or from about 125 nm to about 175 nm.

An implementation of an ODH catalyst material has a mean particle size from about 0.5 μm to about 10 μm, or from about 2 μm to about 8 μm, or from about 3 μm to about 5 μm, or from about 0.5 μm to about 20 μm, or from about 5 μm to about 15 μm, or from about 7 μm to about 11 μm.

An implementation of an ODH catalyst material is characterized by having at least one or more XRD diffraction peaks (2θ degrees) chosen from 6.5±0.2, 7.8±0.2, 8.9±0.2, 10.8±0.2, 13.2±0.2, 14.0±0.2, 22.1±0.2, 23.8±0.2, 25.2±0.2, 26.3±0.2, 26.6±0.2, 27.2±0.2, 27.6±0.2, 28.2±0.2, 29.2±0.2, 30.5±0.2, and 31.4±0.2 wherein the XRD is obtained using CuKα radiation. An implementation of an ODH catalyst material is characterized by having at least one or more XRD diffraction peaks (2θ degrees) chosen from 6.6±0.2, 6.8±0.2, 8.9±0.2, 10.8±0.2, 13.0±0.2, 22.1±0.2, 26.7±0.2, 27.2±0.2, and 28.2±0.2, wherein the XRD is obtained using CuKα radiation.

An implementation of an ODH catalyst material can include from about 0.8 wt. % to about 30 wt. % calcium. The catalyst material can include about 0.15 wt. % to about 2.8 wt. % calcium. The catalyst material can include about 0.5 wt. % to about 75 wt. % calcium carbonate. The catalyst material can include about 5 wt. % to about 15 wt. % calcium carbonate.

The catalyst may be supported on or agglomerated with a binder, carrier, diluent or promoter. Some binders include acidic, basic or neutral binder slurries of TiO₂, ZrO₂, Al₂O₃, AlO(OH) and mixtures thereof. Another useful binder includes Nb₂O₅. The agglomerated catalyst may be extruded in a suitable shape (rings, spheres, saddles, etc.) of a size typically used in fixed bed reactors. When the catalyst is extruded, various extrusion aids known in the art can be used. In some cases, the resulting support may have a cumulative surface area of as high as about 300 m²/g as measured by BET, in some cases less than about 35 m²/g , in some cases, less than about 20 m²/g, in other cases, less than about 3 m²/g, and a cumulative pore volume from about 0.05 to about 0.50 cm³/g.

The catalysts may be alone or in combination. Also, in some embodiments the catalysts may be used with a promotor such ad Bi, Be, Nb, Ta, Ti, Pd, Pt, Re or Ru to increase the catalyst activity.

The mixed metal oxide catalyst can be a supported catalyst. The support may be selected from oxides of titanium, zirconium, aluminum, magnesium, yttrium, lanthanum, silicon, zeolites and clays and their mixed compositions or a carbon matrix. The mixed metal oxide catalyst can also have a binder added which increases cohesion among the catalyst particles and optionally improves adhesion of the catalyst to the support if present. The mixed metal oxide catalyst can be diluted with heat dissipative particles, such as DENSTONE® 99 alumina particles or corrosion resistant steels such as SS 316 particles.

### Oxygen/Alkane Mixture

Mixtures of one or more C2-C4 alkanes (for example from Alkane port 124 in FIG. 1 or Alkane port 224 in FIG. 2) with oxygen (for example from O₂ Enriched Permeate Line 118 in FIG. 1 or O₂ Enriched Permeate Line 218 in FIG. 2) can be employed using ratios that fall outside of the flammability envelope of the one or more C2-C4 alkanes and oxygen. The ratio of C2-C4 alkane to oxygen may fall outside the upper flammability envelope; in these cases, the percentage of oxygen in the mixture can be less than about 30 vol %, in some cases less than about 25 vol %, or in other cases less than about 20 vol %.

In cases with higher oxygen percentages, C2-C4 alkane percentages can be adjusted to keep the mixture outside of the flammability envelope. While a person skilled in the art would be able to determine an appropriate ratio level, in many cases the percentage of C2-C4 alkane is less than about 40 vol %. As a non-limiting example, where the mixture of gases prior to the ODH process includes about 10 vol % oxygen and about 15 vol % C2-C4 alkane, the balance can be made up with a heat removal diluent gas. Non-limiting examples of useful heat removal diluent gas in this embodiment include, but are not limited to, one or more of nitrogen, carbon dioxide, and steam. In some embodiments, the heat removal diluent gas should exist in the gaseous state at the conditions within the reactor and should not increase the flammability of the hydrocarbon added to the reactor, characteristics that a skilled worker would understand when deciding on which heat removal diluent gas to employ. The heat removal diluent gas can be added to either of the C2-C4 alkane containing gas or the oxygen containing gas or to both gases prior to entering the ODH reactor (for example ODH Reactor 102 in FIG. 1 or ODH Reactor 202 in FIG. 2) or may be added directly into the ODH reactor.

In some embodiments mixtures that fall within the flammability envelope may be employed, as a non-limiting example, in instances where the mixture exists in conditions that prevent propagation of an explosive event. In these non-limiting examples, the flammable mixture is created within a medium where ignition is immediately quenched. As a further non-limiting example, a user may design a reactor where oxygen and the one or more C2-C4 alkanes are mixed at a point where they are surrounded by a flame arresting material. Any ignition would be quenched by the surrounding material. Flame arresting materials include, but are not limited to, metallic or ceramic components, such as stainless steel walls or ceramic supports. In some embodiments, oxygen and C2-C4 alkane can be mixed at a low temperature, where an ignition event would not lead to an explosion, then introduced into the reactor before increasing the temperature. Flammable conditions may not exist when the mixture is surrounded by the flame arrestor material inside of the reactor.

### Carbon Monoxide Output

Carbon monoxide can be produced in the ODH reaction as a by-product of oxidation of the one or more C2-C4 alkanes. The carbon monoxide output is a function of the amount of carbon monoxide produced in the oxidative process.

Measuring the amount of carbon monoxide coming off the ODH reactor can be done using any means known in the art. For example, one or more detectors such as gas chromatography (GC), infrared spectroscopy (IR), or Raman spectroscopy detectors, are situated immediately downstream of the reactor to measure the carbon monoxide output. While not required, the output of other components may also be measured. These include but are not limited to the amounts of ethylene, unreacted ethane, carbon dioxide and oxygen, and by-products such as acetic acid.

Carbon monoxide output can be stated using any metric commonly used in the art. For example, the carbon monoxide output can be described in terms of mass flow rate (g/min) or volumetric flow rate (cm³/min). In some embodiments, normalized selectivity can be used to assess the degree to which carbon monoxide is produced or consumed. In that instance the net mass flow rate of CO (i.e. the difference between the mass flow rate of CO leaving the ODH reactor and the mass flow rate of CO entering the reactor) is normalized to the conversion of ethane, in essence describing what fraction of ethane is converted into carbon monoxide as opposed to ethylene, or other by-products such as acetic acid.

Many industrial processes, in addition to ODH, produce carbon monoxide which must be captured or flared where it contributes to the emission of greenhouse gases. Using the carbon monoxide mitigation steps disclosed herein converts most, if not all, carbon monoxide resulting from the ODH process to carbon dioxide. An advantage then is the ability to reduce or eliminate the amount of carbon monoxide produced in the ODH process in combination with other processes, such as thermal cracking.

### Acetylene Output

Acetylene can be produced in the ODH reaction as a by-product of oxidation of the one or more C2-C4 alkanes. The acetylene output is a function of the amount of acetylene produced in the oxidative process.

Measuring the amount of acetylene coming off the ODH reactor can be done using any means known in the art. For example, one or more detectors such as GC, IR, or Raman detectors, are situated immediately downstream of the reactor to measure the acetylene output. While not required, the output of other components may also be measured. These include but are not limited to the amounts of ethylene, unreacted ethane, carbon monoxide, carbon dioxide and oxygen, and by-products such as acetic acid.

Acetylene output can be stated using any metric commonly used in the art. For example, the acetylene output can be described in terms of mass flow rate (g/min), volumetric flow rate (cm³/min) or volumetric parts per million (ppmv). In some embodiments, normalized selectivity can be used to assess the degree to which acetylene is produced or consumed. In that instance the net mass flow rate of acetylene (i.e. the difference between the mass flow rate of acetylene leaving the ODH reactor and the mass flow rate of acetylene entering the reactor) is normalized to the conversion of ethane, in essence describing what fraction of ethane is converted into acetylene as opposed to ethylene, or other by-products such as acetic acid.

Using the acetylene mitigation steps disclosed herein reacts most, if not all, acetylene resulting from the ODH process. An advantage then is the ability to reduce or eliminate the amount of acetylene produced in the ODH process in combination with other processes, such as thermal cracking and eliminate downstream unit operations in an ODH-type process.

### Addition of Steam

The amount of steam added to the ODH process affects the degree to which carbon dioxide acts as an oxidizing agent. In some embodiments steam may be added directly to the ODH reactor, or steam may be added to the individual reactant components (i.e. the C2-C4 alkane, oxygen, or heat removal diluent gas) or combinations thereof, and subsequently introduced into the ODH reactor along with one or more of the reactant components. Alternatively, steam may be added indirectly as water mixed with either the C2-C4 alkane, oxygen or heat removal diluent gas, or a combination thereof, with the resulting mixture being preheated before entering the reactor. When adding steam indirectly as water the preheating process should increase the temperature so that the water is entirely converted to steam before entering the reactor.

Increasing the amount of steam added to a reactor increases the degree to which carbon dioxide acts as an oxidizing agent. Decreasing the amount of steam added to the reactor decreases the degree to which carbon dioxide acts as an oxidizing agent. In some embodiments a user monitors the carbon dioxide output and compares it to a predetermined target carbon dioxide output. If the carbon dioxide output is above the target a user can then increase the amount of steam added to the ODH process. If the carbon dioxide output is below the target a user can decrease the amount of steam added to the ODH process, provided steam has been added. Setting a target carbon dioxide output level is dependent on the requirements for the user. In some embodiments increasing the steam added will have the added effect of increasing the amount of acetic acid and other by-products produced in the process. A user that is ill-equipped to separate out larger amounts of acetic acid from the output of the ODH process may instead reduce steam levels to a minimum, while a user that desires a process that consumes carbon dioxide may choose to maximize the amount of steam that can be added. The amount of steam added to the one or more ODH reactors can be up to about 80 vol %, in some cases up to about 60 vol %, in some cases up to about 40 vol %, in some cases up to about 35 vol %, in other cases up to about 30 vol %, and in some instances up to about 25 vol %.

In some embodiments when using two or more ODH reactors a user may choose to control carbon dioxide output in only one, or less than the whole complement of reactors. For example, a user may opt to maximize carbon dioxide output of an upstream reactor so that the higher level of carbon dioxide can be part of the heat removal diluent gas for the subsequent reactor. In that instance, maximizing carbon dioxide output upstream minimizes the amount of heat removal diluent gas that would need to be added to the stream prior to the next reactor.

There is no requirement for adding steam to an ODH process, as it is one of many alternatives for the heat removal diluent gas. For processes where no steam is added, the carbon dioxide output is maximized under the conditions used with respect to ethane, oxygen and heat removal diluent gas inputs. Decreasing the carbon dioxide output is then a matter of adding steam to the reaction until carbon dioxide output drops to the desired level. In embodiments where oxidative dehydrogenation conditions do not include addition of steam, and the carbon dioxide output is higher than the desired carbon dioxide target level, steam may be introduced into the reactor while keeping relative amounts of the main reactants (i.e. C2-C4 alkane and oxygen) and heat removal diluent gas added to the reactor constant, and monitoring the carbon dioxide output, increasing the amount of steam until carbon dioxide decreases to the target level.

A carbon dioxide neutral process can be achieved by increasing steam added so that any carbon dioxide produced in the ODH process can then be used as an oxidizing agent such that there is no net production of carbon dioxide. Conversely, if a user desires net positive carbon dioxide output then the amount of steam added to the process can be reduced or eliminated to maximize carbon dioxide production. As the carbon dioxide levels increase there is potential to reduce oxygen consumption, as carbon dioxide is competing as an oxidizing agent. The skilled person would understand that using steam to increase the degree to which carbon dioxide acts as an oxidizing agent can impact oxygen consumption. The implication is that a user can optimize reaction conditions with lower oxygen contributions, which may assist in keeping mixtures outside of flammability limits.

The stream exiting the one or more ODH reactors can be treated to remove or separate water and water-soluble hydrocarbons from the stream exiting the one or more ODH reactors. This stream can be fed to a CO Oxidation reactor.

### Acetic Acid Removal

Prior to being fed to a CO Oxidation Reactor, the stream exiting the one or more ODH reactors can be directed to quench tower or acetic acid scrubber, for example Quench Tower 104 in FIG. 1 or Quench Tower 204 in FIG. 2, which facilitates removal of oxygenates, such as acetic acid, ethanol, and water via a bottom outlet, for example Quench Tower Bottom Outlet 126 or Quench Tower Bottom Outlet 226. A stream containing unconverted C2-C4 alkane (such as ethane), corresponding C2-C4 alkene (such as ethylene), unreacted oxygen, carbon dioxide, carbon monoxide, optionally acetylene and heat removal diluent gas, are allowed to exit the scrubber via, for example Quench Tower Overhead 128 or Quench Tower Overhead 228.

The oxygenates removed via for example Quench Tower 104 or Quench Tower 204 or acetic acid scrubber can include carboxylic acids (for example acetic acid), aldehydes (for example acetaldehyde), alcohol (for example ethanol) and ketones (for example acetone). The amount of oxygenate compounds remaining in the stream exiting the scrubber via for example Quench Tower Overhead 128 or Quench Tower Overhead 228 will often be zero, i.e, below the detection limit for analytical test methods typically used to detect such compounds. When oxygenates can be detected they can be present at a level of up to about 1 per million by volume (ppmv), in some cases up to about 5 ppmv, in other cases less than about 10 ppmv, in some instances up to about 50 ppmv and in other instances up to about 100 ppmv and can be present up to about 1,000 ppmv, in some cases up to about 1 vol %, in other cases up to about 2 vol %. The amount of oxygenates or acetic acid in the stream exiting the scrubber via for example Quench Tower Overhead 128 or Quench Tower Overhead 228 can be any value, or range between any of the values recited above.

### Removal of Oxygen

Carbon monoxide, oxygen and acetylene are contaminants, that can affect the performance of equipment downstream of the one or more ODH reactors and/or have a negative impact on the purity of the final ethylene product.

Optionally, an Oxygen Removal Vessel could take the, for example, Quench Tower Overhead 128 or Quench Tower Overhead 228. A reactor placed downstream of the one or more ODH reactors containing a catalyst material that includes CuO and ZnO can remove all or part of the carbon monoxide, oxygen and acetylene in the process stream passing through. In some embodiments, the material that includes CuO and ZnO can act as an adsorbent for carbon monoxide, oxygen and acetylene. In other embodiments, the material that includes CuO and ZnO can perform as a selective carbon monoxide oxidation catalyst.

In some embodiments, after a bed of material that includes CuO and ZnO is depleted of chemosorbed oxygen the material can initiate a chemical reaction whereby oxygen and acetylene are removed or eliminated, without removing carbon monoxide from the process stream. Not being limited by any single theory, it is believed that in this embodiment, CuO and ZnO are reduced to their corresponding elemental metal forms via the reaction.

When the above described reactor containing a catalyst material that includes CuO and ZnO is placed downstream of the one or more ODH reactors, the mode of operation can be beneficial in certain integration options of ODH with different plants where carbon monoxide can be a feedstock that is often used for downstream plants as compared to carbon dioxide.

### Amine Wash

A separation method applicable for use with the present disclosure is the use of alkylamines, referred to herein as amines, in a scrubber to remove carbon dioxide from gaseous compositions, as shown as Amine Wash Tower 108 in FIG. 1 or Amine Wash Tower 208 in FIG. 2. Carbon dioxide present in a gas can be absorbed by an aqueous amine solution, which can then be separated from the remaining gaseous components. The amine containing solution can be stripped of carbon dioxide by heating the solution above about 100° C. and recycling to continue the process. The Amine Wash Tower may be operated at a pressure from about 650 kPa to about 1100 kPa, which may require a compressor upstream of the tower. The carbon dioxide, which is typically highly concentrated, can be captured and sold, or, alternatively it can be recycled back to act as a heat removal diluent gas for the C2-C4 alkane and oxygen containing gases when introduced into an ODH Reactor, such as ODH Reactor 102 or ODH Reactor 202. Carbon dioxide produced in the process can be captured instead of being flared where it contributes to greenhouse gas emissions.

Consideration of the type of amines used in the process requires special attention. The particular amines that are used vary in their ability to remove carbon dioxide and in their tendency to promote the formation of degradation products. As a non-limiting example, monoethanolamine (MEA) is commonly used and is capable of removing a high percentage of carbon dioxide, even at low concentrations, but can also react with the carbon dioxide to form degradation products. This results in lower carbon dioxide capture and a reduction of available amines for subsequent absorption cycles.

### Oxidation of Carbon Monoxide

Oxygen can also be removed reacting it with carbon monoxide to form carbon dioxide. In this instance, the reactor product, for example the contents in ODH Reactor Product Line 122 or ODH Reactor Product Line 222, is fed to a CO Oxidation reactor (not shown), which can contain a catalyst that includes one or more selected from a group 11 metal, a group 4 metal, a group 7, a group 9 metal, a lanthanide metal, and an actinide metal and/or their corresponding metal oxides capable of converting at least a portion of the carbon monoxide to carbon dioxide. The carbon dioxide can be recycled to an ODH Reactor, for example ODH Reactor 102 or ODH Reactor 202, as described herein.

The group 11 metal can be selected from copper, silver, gold and combinations thereof. The group 4 metal can be selected from titanium, zirconium, hafnium, rutherfordium and combinations thereof. The group 7 metal can be selected from manganese, technetium, rhenium, bohrium and combinations thereof. In embodiments of the disclosure, the group 9 metal can be selected from cobalt, rhodium, iridium, meitnerium and combinations thereof. The lanthanide metal can be selected from La, Ce, Pr, Nd, Pm, Sm, Eu, Gd, Tb, Dy, Ho, Er, Tm, Yb and combinations thereof. The actinide metal can be selected from Ac, Th, Ps, U, Np, Pu, Am, Cm, Bk, Cf, Es, Fm, Md, No and combinations thereof.

The CO Oxidation reactor catalyst, in some cases a group 11 metal, can be used in conjunction with a promoter. The promoter can be selected from one or more of the lanthanide and actinide metals (as defined above) and their corresponding metal oxides. The promoter can be selected from one or more of the lanthanide metals and their corresponding metal oxides. The promoter can include cerium and its corresponding metal oxides.

The CO Oxidation reactor catalyst, in some cases a group 11 metal, and optional promotor can be provided on a support. The support is typically an inert solid with a high surface area, to which the CO Oxidation reactor catalyst and optional promotor can be affixed. The support can include Si, Ge, Sn, their corresponding oxides and combinations thereof.

Non-limiting examples of suitable CO Oxidation reactor catalysts with optional promotors and supports include Ag/SiO₂, AgCeO₂/SiO₂, AgZrO₂/SiO₂, AgCo₃O₄/SiO₂, Cu/SiO₂, CuCeO₂/SiO₂, CuZrO₂/SiO₂, CuCo₃O₄/SiO₂ and combinations thereof. Non-limiting examples of suitable CO Oxidation reactor catalysts with optional promotors and supports include AgCeO₂/SiO₂, AgZrO₂/SiO₂ and combinations thereof.

The CO Oxidation reactor catalyst can include silver, the optional promoter can include cerium and the support can include SiO₂.

The CO Oxidation reactor catalyst can include copper, the optional promoter can include cerium and the support can include SiO₂.

When oxidation of carbon monoxide is preferentially desired, the CO Oxidation reactor catalyst can include manganese, the optional promoter can include cerium and the support can include SiO₂.

### Acetylene Oxidation

Another non-limiting example of a reaction that can remove oxygen is oxidation of acetylene. In this non-limiting example, the reactor product, for example the contents in ODH Reactor Product Line 122 or ODH Reactor Product Line 222, is fed to the CO Oxidation reactor (not shown), which can contain a catalyst that can include one or more selected from a group 11 metal, a group 4 metal, a group 9 metal, a lanthanide metal, and an actinide metal and/or their corresponding metal oxides capable of reacting at least a portion of the acetylene.

The group 11 metal can be selected from copper, silver, gold and combinations thereof. The group 4 metal can be selected from titanium, zirconium, hafnium, rutherfordium and combinations thereof. The group 9 metal can be selected from cobalt, rhodium, iridium, meiternium and combinations thereof. The lanthanide metal can be selected from La, Ce, Pr, Nd, Pm, Sm, Eu, Gd, Tb, Dy, Ho, Er, Tm, Yb and combinations thereof. The actinide metal can be selected from Ac, Th, Ps, U, Np, Pu, Am, Cm, Bk, Cf, Es, Fm, Md, No and combinations thereof.

The CO Oxidation reactor catalyst, in some cases a group 11 metal, can be used in conjunction with a promoter. The promoter can be selected from one or more of the lanthanide and actinide metals (as defined above) and their corresponding metal oxides. The promoter can be selected from one or more of the lanthanide metals and their corresponding metal oxides. The promoter can include cerium and its corresponding metal oxides.

The CO Oxidation reactor catalyst, in some cases a group 11 metal, and optional promotor can be provided on a support. The support is typically an inert solid with a high surface area, to which the CO Oxidation reactor catalyst and optional promotor can be affixed. The support can include Si, Ge, Sn, their corresponding oxides and combinations thereof.

Non-limiting examples of suitable CO Oxidation reactor catalysts with optional promotors and supports can include Ag/SiO₂, AgCeO₂/SiO₂, AgZrO₂/SiO₂, AgCo₃O₄/SiO₂, Cu/SiO₂, CuCeO₂/SiO₂, CuZrO₂/SiO₂, CuCo₃O₄/SiO₂ and combinations thereof.

Non-limiting examples of suitable CO Oxidation reactor catalysts with optional promotors and supports can include AgCeO₂/SiO₂, AgZrO₂/SiO₂ and combinations thereof.

### Caustic Wash Tower

A “caustic wash tower”, “scrubber” or “wet scrubber” is typically a large-scale treatment unit that performs a continuous wash by spraying the ODH process stream with a caustic absorption liquid. As a non-limiting example, the caustic wash tower can be used to purify the ODH process stream to remove, as non-limiting examples, acid gases such as hydrogen sulphide (H₂S) and carbon dioxide (CO₂). A caustic wash tower could optionally be placed after the amine wash tower, for example Amine Wash Tower 108 or Amine Wash Tower 208.

### Demethanizer Distillation Tower

The demethanizer Distillation Tower 110 typically includes a cryogenic distillation column. The distillate Overhead Stream 140 is a combination of methane and lighter gases, that can include hydrogen, CO, and nitrogen gas. The remaining liquid in C2/C2+ Hydrocarbons Bottom Outlet 138 includes higher hydrocarbons.

In an embodiment of this disclosure the Distillation Tower 110 includes an outlet for removal of the Overhead Stream 140 and a C2/C2+ Hydrocarbons Bottom Outlet 138 for removal of the C2/C2+ hydrocarbon. In another embodiment of this disclosure the distillation tower includes a side outlet for removal of C2-C4 alkenes.

A compressor and/or a heat exchanger may be required upstream of a demethanizer distillation tower.

### C2/C2+ Distillation Tower

It is well known that the degree of separation capable within a distillation tower is dependent upon the number of trays within the unit. The most common method involves cryogenic distillation so the nature of the species targeted for separation and their relative volatilities plays a role. For example, the relative volatility of ethylene to ethane is quite small. As a result, a tower designed to separate the two species needs to be tall and include a large number of trays. The C2/C2+ Hydrocarbons Bottom Outlet 138 can be directed to a C2+ splitter (not shown) to separate the C2-C4 alkane from its corresponding C2-C4 alkene. The C2-C4 alkane can be fed back to the ODH reactor, and the corresponding C2-C4 alkene, the target product, can be captured and employed for use in a variety of applications that depend on the nature of the C2-C4 alkene. For example, if the desired product is ethylene then use in synthesis of polyethylene would be appropriate.

A compressor and/or a heat exchanger may be required upstream of a C2/C2+ distillation tower.

### Oxygen Separation Module

An example of an Oxygen Separation Module 148 in FIG. 1 or Oxygen Separation Module 246 in FIG. 2, is a sealed vessel with two compartments, separated by a temperature dependent Oxygen Transport Membrane 150 or Oxygen Transport Membrane 248. The two compartments are the Retentate Side 114 or Retentate Side 214, and the Permeate Side 112 or Permeate Side 212. That the membrane is temperature dependent means that at a critical temperature the membrane will selectively allow oxygen to pass through from one side to the other.

There are two outputs from the Oxygen Separation Module, an O₂ Depleted Air Exhaust Line 146 or O₂ Depleted Air Exhaust Line 244 for removal of oxygen depleted air and combustion products from the Retentate Side 114, and an outlet for removal of oxygen, O₂ Enriched Permeate Line 118 or O₂ Enriched Permeate Line 218, and possibly combustion products from the Permeate Side 112.

In an embodiment of this disclosure, the O₂ enriched permeate, and possibly combustion products, may be recycled back as or part of the Oxygen introduced into the ODH Reactor.

In an embodiment of this disclosure, the oxygen depleted air exhaust may be recycled to the Combustion Chamber.

In an embodiment of this disclosure, the Oxygen Transport Membrane 150 from FIG. 1, or Oxygen Transport Membrane 248 from FIG. 2, is a tube that divides a Permeate Side from a Retentate Side. Material suitable for construction of the outer wall of the Retentate Side include those resistant to temperatures that exceed about 850° C. and approach about 1000° C., selection of which falls within the knowledge of the skilled worker.

The present disclosure contemplates the Overhead Stream 140 or Overhead Stream 240 entering a Combustion Chamber 106 or Combustion Chamber 206 which can provide heat by the combustion of fuel from Fuel Line 152 or Fuel Line 252. In an embodiment of this disclosure, an outlet of a Combustion Chamber, Flue Gas, can be directed to an Oxygen Separation Module into either of a Permeate Side or a Retentate Side. This disclosure also contemplates the use of a valve for switching between directing the Flue Gas to the Retentate Side or the Permeate Side. This would allow an operator to choose which of the sides, permeate or retentate, that the overhead stream is directed to.

The flue gas (for example the contents of Flue Gas Line 116 or Flue Gas Line 254) is the energy carrier for the Oxygen Separation Module. The flue gas can transfer the energy directly or indirectly. If done directly, the flue gas is directed to the Oxygen Separation Module and mixed either with permeate or retentate right at the unit inlet. If done indirectly, the flue gas it heats up the air outside the Oxygen Separation Module.

CO₂ can be used as one of the diluents—the combustion can operate on CO₂ and O₂ instead of air. This would allow converting CO₂ in the ODH reactor to products such as acetic acid.

The present disclosure also contemplates introducing a Flue Gas into both the Retentate Side and Permeate Side simultaneously. This includes the ability to alter the relative amount of Flue Gas which is entered into each side. For example, an operator may choose to permit about 80% of the Flue Gas to enter into the Retentate Side and only about 20% to the Permeate Side, or vice versa. To be clear, the amount of the Flue Gas that enters either side, permeate or retentate, can range from about 0 to about 100%, with the fraction for each side totaling 100%. Precision valves that can control the flow sent to either side are well known in the art, and include, without limitation, solenoid valves, ball valves, or a combination of a backpressure needle valve and solenoid valve.

The present disclosure also contemplates introducing a feed air stream via Feed Air Line 142 or Feed Air Line 242 into both the Retentate Side and Permeate Side simultaneously. This includes the ability to alter the relative amount of feed air which is entered into each side. For example, an operator may choose to permit 80% of feed air to enter into the Retentate Side and only 20% to the Permeate Side, or vice versa. To be clear, the amount of the feed air stream that enters either side, permeate or retentate, can range from about 0 to about 100%, with the fraction for each side totaling 100%. Precision valves that can control the flow sent to either side are well known in the art, and include, without limitation, solenoid valves, ball valves, or a combination of a backpressure needle valve and solenoid valve.

The Oxygen Transport Membrane component of the Oxygen Separation Module selectively allows passage of oxygen when the membrane reaches a critical temperature. Membranes of this nature are known. Specifically, a Mixed Ionic-Electronic Conducting (MIEC) membrane is contemplated for use in this disclosure. Movement of oxygen across the Oxygen Transport Membrane is driven by an oxygen partial pressure gradient, moving from the high oxygen partial pressure side to the low oxygen partial pressure side. To get the oxygen to move to the Permeate Side, a skilled operator would understand that the partial pressure of oxygen on the Retentate Side would need to be increased to the point where it equals or exceeds the partial pressure of oxygen on the Permeate Side. For example, if oxygen on the Permeate Side is close to 100% of the volume at a pressure of about 1 atm, then the pressure on the Retentate Side would need to be increased to at least about 5 atm when atmospheric air is added and contains approximately 21% oxygen by volume. Alternatively, the pressure on the Permeate Side could be reduced to levels at or below about 0.2 atm using a vacuum driven process.

Also contemplated in the design of the Oxygen Separation Module is the ability to add a sweep gas (not shown), such as steam or carbon dioxide, to the Permeate Side to dilute oxygen that crosses over from the Retentate Side. The effect of the sweep gas is the lowering of the oxygen partial pressure on the Permeate Side to drive diffusion of oxygen from the Retentate Side. A result of this configuration is a much lower percentage of oxygen within the oxygen enriched permeate, as it is diluted by the sweep gas. Theoretically, the oxygen percentage could drop well below 10%. However, if water is the sweep gas, then a heat exchanger downstream of Oxygen Separation Module can be used to remove the water following condensation, increasing the relative amount of oxygen in the line. If carbon dioxide is used, then an operator can determine the amount required to produce the desired oxygen level in the oxygen enriched permeate. By altering the amount of sweep gas an operator can control how much oxygen is present in the line as it leaves the Oxygen Separation Module. A person skilled person in the art would understand this relationship and would be familiar with using a sweep gas and with using means for controlling the pressure in a sealed vessel, such as, the type contemplated for the Oxygen Separation Module described in this disclosure.

The oxygen flux across the Oxygen Transport Membrane is dependent upon the thickness of the membrane. A thin membrane allows oxygen to cross more quickly than a thick membrane. A membrane that includes a single layer, or monolithic type membrane, may be reduced in thicknesses in the range of about 0.1 to about 0.2 μm to allow greater oxygen flux. However, these thicknesses are not practical due to susceptibility to mechanical instability. If a monolithic membrane is to be used, thicknesses below about 0.2 mm are not recommended. Other known membrane configurations include asymmetric membranes where a very thin conducting layer is supported on both sides by a porous structure. This allows a user to employ very thin membranes that allow higher oxygen flux without sacrificing stability. It is not essential to use any particular membrane structure provided the oxygen flux across the membrane is sufficient. In the present disclosure the Oxygen Transport Membrane has an oxygen flux within the range of about 300 to about 1500 l/hr*m², more in some cases from about 500 to about 1300 l/hr*m², and in other cases from about 700 to about 1000 l/hr*m².

During start-up of the chemical complex, the Oxygen Transport Membrane may not be at the required temperature. As a result, oxygen from the injected Air Input cannot pass into the Permeate Side. In this instance, it is often desirable to direct the Flue Gas solely into the Retentate Side so that combustion on that side can contribute to increasing the temperature of the Oxygen Transport Membrane to the point where oxygen can cross. When at steady state and the temperature of the Oxygen Transport Membrane exceeds about 850° C., the Overhead Stream may be directed to either side because oxygen can freely pass and permit combustion such that heat is continuously generated. Alternatively, during startup, other means, such as a heated feed air, may be used to heat the Oxygen Transport Membrane.

The Oxygen Transport Membrane described above is susceptible to unintentional hot spots, which could damage or destroy a membrane, or any upsets within a membrane unit which could lead to temporary extinguishing of the flame and possibly leading to an internal explosion and potentially damaging a membrane. The configuration described above may also require a membrane unit designed to deflagration containment design requirements.

### Hot Gas Supplied by External Combustion

The process in this disclosure can involve heating an Oxygen Transport Membrane with a hot gas provided by an external combustion chamber, which can supply heat either on a Permeate Side or a Retentate Side or both of the membrane unit.

A stream, shown in FIG. 1 in O₂ Burn Line 154, and FIG. 2 in O₂ Burn Line 250, can be formed as part of a stream of oxygen enriched permeate, and can be heated by combustion in an external Combustion Chamber; a hot gas is then a Flue Gas of the Combustion Chamber. A Flue Gas can then be fed in whole or in part to an Oxygen Transport Membrane. Heat to an Oxygen Transport Membrane can be supplied by external combustion in a Combustion Chamber of one of the gases going to the Oxygen Transport Membrane using any of the following configurations:
1. An external device can supply heat to the Permeate Side 112 of the membrane as shown in FIG. 1. Some of the contents in O₂ Enriched Permeate Line 118 can be directly sent to the external furnace or Combustion Chamber 106 via O₂ Burn Line 154, where it can combust fuel from Fuel Line 152 and Overhead Stream 140, increase the temperature to the desired level and enter the Oxygen Separation Module 148 as flue gas from Flue Gas Line 116 providing heat to the Oxygen Transport Membrane 150.
2. An external device can supply heat to the Retentate Side 214 of the membrane as shown in FIG. 2. This can involve heating at least part of the contents in O₂ Depleted Air Exhaust Line 244 and potentially some other source of O₂ to a temperature exceeding autoignition temperature of the fuel in Fuel Line 252 and Overhead Stream 240 being used, and sending the hot Air Input 216 to the Retentate Side 214 of the Oxygen Separation Module 246. The hot flue gas in Flue Gas Line 254 can also be used to indirectly heat the feed air in Feed Air Line 242 entering the Oxygen Separation Module 246.
3. A combination of external devices can supply heat to the permeate side and/or the retentate side of the oxygen transport membrane.

There is no need for deflagration containment with an external combustion chamber. If the external combustion chamber supplies heat to the permeate side of an oxygen transport membrane, the permeate side can operate with low pressure, such as atmospheric pressure or lower. The flue gas can also play a role of sweep gas on the permeate side of an oxygen transport membrane to reduce the partial pressure of O₂ on the permeate side and increase the membrane efficiency, without the need to create a vacuum.

The process conditions desired for the flue gas in Flue Gas Line 116 can be determined by the process conditions desired of the Overhead Stream 140 prior to entering the Oxygen Separation Module 148.

The temperature of the flue gas in Flue Gas Line 116 can be about 850° C. to about 1500° C., or about 860° C. to about 1400° C., or about 870° C. to about 1300° C., or about 880° C. to about 1200° C.

The pressure inside the Combustion Chamber 106 can be atmospheric to about 700 kPag, or about 10 kPag to about 650 kPag, or about 20 kPag to about 600 kPag.

The fuel in Fuel Line 152 stream that is fed to the Combustion Chamber 106 can be a combustible fuel and can include a hydrocarbon, such as methane, ethane, propane or a mixture of hydrocarbons such as natural gas. The fuel in Fuel Line 152 stream can include nitrogen, water, CO₂ and CO.

The ratios of the mass flows of the stream in O₂ Burn Line 154 to the stream in O₂ Enriched Permeate Line 118 can be from about 0 to about 1, or from about 0.1 to about 0.9, or from about 0.2 to about 0.8.

This disclosure will further be described by reference to the following examples. The following examples are merely illustrative of this disclosure and are not intended to be limiting. Unless otherwise indicated, all percentages are by weight.

### EXAMPLES

### Example 1

A simulation of an ODH reactor was developed using gPROMS ProcessBuilder® (Process Systems Enterprise Limited) 1.2.0. The SRK equation of state was used to define component properties in Multiflash, advanced thermodynamics software (KBC). The kinetic model for the ODH reaction was developed in gPROMS ProcessBuilder® 1.2.0 and the kinetic parameters were estimated using some fixed bed reactor data. The mixed metal oxide catalyst used was MoₐV_{b}Te_{c}Nb_{d}Oₑ, wherein a, b, c, d, and e are the relative atomic amounts of the elements Mo, V, Te, Nb, and O, respectively; and when a=1, b=0.01 to 1.0, c=0.01 to 1.0, d=0.01 to 1.0, and e is a number to satisfy the valence state of the catalyst. The model predictions are in good agreement with the reactor data and are shown in Table 1 and Table 2.

TABLE 1
ODH Reactor Operating Conditions, Catalyst
Activity and Product Distribution

| | | |
|---|---|---|
| Ethane Conversion | 64.89 | % |
| Ethylene Selectivity | 77.55 | % |
| CO₂ Selectivity | 7.62 | % |
| CO Selectivity | 9.56 | % |
| Acetic Acid Selectivity | 5.27 | % |
| GHSV | 2533.941 | h⁻¹ |
| Reactor Average Temperature | 384 | ° C. |
| Reactor Inlet Pressure | 267 | bar |

TABLE 2
ODH Reactor Feed and Product Mass Balance

| | Feed to | Product from | |
|---|---|---|---|
| | Reactor | Reactor | Units |
| Mass Flow Rates (×10⁻⁶) | | | |
| C₂H₆ | 26.2 | 9.19 | kg/s |
| C₂H₄ | 0 | 12.3 | kg/s |
| CO₂ | 0 | 3.79 | kg/s |
| CO | 0 | 3.03 | kg/s |
| H₂O | 77.8 | 91.5 | kg/s |
| CH₃COOH | 0 | 1.79 | kg/s |
| O₂ | 18.3 | 0.762 | kg/s |
| Total | 122 | 122 | kg/s |
| Molar Flow Rates (×10⁻³) | | | |
| C₂H₆ | 3.13 | 1.10 | kmol/h |
| C₂H₄ | 0 | 1.58 | kmol/h |
| CO₂ | 0 | 0.310 | kmol/h |
| CO | 0 | 0.389 | kmol/h |
| H₂O | 15.6 | 18.3 | kmol/h |
| CH₃COOH | 0 | 0.107 | kmol/h |
| O₂ | 2.08 | 0.0857 | kmol/h |
| Total | 20.7 | 21.9 | kmol/h |
| Molar Fractions | | | |
| C₂H₆ | 0.151 | 0.0503 | mol/mol |
| C₂H₄ | 0 | 0.0721 | mol/mol |
| CO₂ | 0 | 0.0142 | mol/mol |
| CO | 0 | 0.0178 | mol/mol |
| H₂O | 0.750 | 0.837 | mol/mol |
| CH₃COOH | 0 | 0.00490 | mol/mol |
| O₂ | 0.0994 | 0.00392 | mol/mol |
| Pressure | 2.67 | 2.65 | bar |
| Temperature | 25.2 | 370 | ° C. |

The results of Example 1 were used to identify an example of how much O₂ (2.08×10⁻³ kmol/h) can be required to be generated in an oxygen transport membrane unit to be fed to an ODH reactor, and an example of how much CO (0.389 mol/h) is available in the product stream of this ODH reactor to be sent to a combustion chamber prior to an oxygen transport membrane unit.

### Example 2

### Direct Mixing of Flue Gas and Air Going to Oxygen Separation Module

Simulations were developed using AspenPlus® software V10 (Aspen Technology, Inc.). The configuration shown in FIG. 3 was simulated, using the Peng-Robinson equation of state. Steam properties were calculated using STEAMNBS (in AspenPlus). The stream results are shown in Table 3.

TABLE 3
AspenPlus Stream Results for Example 2

| Stream Number | | 304 | 308 | 312 | 316 | 318 | 322 |
|---|---|---|---|---|---|---|---|
| Temperature | ° C. | 850 | 25.0 | 23.8 | 23.8 | 23.8 | 47.3 |
| Pressure | kPa | 545 | 530 | 150 | 150 | 150 | 150 |
| Molar Vapor | | 1.00 | 1.00 | 1.00 | 1.00 | 1.00 | 1.00 |
| Fraction | | | | | | | |
| Mass Density | kg/cum | 1.87 | 6.87 | 1.95 | 1.95 | 1.95 | 1.62 |
| Enthalpy Flow | kJ/hr | 69.7 | 0 | 0 | 0 | 0 | −367 |
| Average MW | | 32.00 | 32.00 | 32.00 | 32.00 | 32.00 | 28.74 |
| Mass Flows | kg/hr | 0.0825 | 0.0825 | 0.0825 | 0.0666 | 0.0159 | 0.413 |
| O₂ | kg/hr | 0.0825 | 0.0825 | 0.0825 | 0.0666 | 0.0159 | 0.0159 |
| N₂ | kg/hr | 0 | 0 | 0 | 0 | 0 | 0.345 |
| H₂O | kg/hr | 0 | 0 | 0 | 0 | 0 | 0.00486 |
| CO₂ | kg/hr | 0 | 0 | 0 | 0 | 0 | 0.0291 |
| CO | kg/hr | 0 | 0 | 0 | 0 | 0 | 0.0109 |
| CH₄ | kg/hr | 0 | 0 | 0 | 0 | 0 | 0.00154 |
| C₂H₆ | kg/hr | 0 | 0 | 0 | 0 | 0 | 9.75E−05 |
| C₃H₈ | kg/hr | 0 | 0 | 0 | 0 | 0 | 8.92E−06 |
| ARGON | kg/hr | 0 | 0 | 0 | 0 | 0 | 0.00585 |
| Mass Fractions | | | | | | | |
| O₂ | | 1.00 | 1.00 | 1.00 | 1.00 | 1.00 | 0.0385 |
| N₂ | | 0 | 0 | 0 | 0 | 0 | 0.835 |
| H₂O | | 0 | 0 | 0 | 0 | 0 | 0.0118 |
| CO₂ | | 0 | 0 | 0 | 0 | 0 | 0.0706 |
| CO | | 0 | 0 | 0 | 0 | 0 | 0.0264 |
| CH₄ | | 0 | 0 | 0 | 0 | 0 | 0.00373 |
| C₂H₆ | | 0 | 0 | 0 | 0 | 0 | 0.000236 |
| C₃H₈ | | 0 | 0 | 0 | 0 | 0 | 2.16E−05 |
| ARGON | | 0 | 0 | 0 | 0 | 0 | 0.0142 |

| Stream Number | | 326 | 330 | 334 | 336 | 340 |
|---|---|---|---|---|---|---|
| Temperature | ° C. | 481 | 928 | 850 | 25.0 | 298 |
| Pressure | kPa | 130 | 545 | 545 | 100 | 560 |
| Molar Vapor | | 1.00 | 1.00 | 1.00 | 1.00 | 1.00 |
| Fraction | | | | | | |
| Mass Density | kg/cum | 0.60 | 1.59 | 1.69 | 1.17 | 3.41 |
| Enthalpy Flow | kJ/hr | −367 | −148 | 115 | −1.96 | 93.4 |
| Average MW | | 29.13 | 29.13 | 29.06 | 28.97 | 28.97 |
| Mass Flows | kg/hr | 0.413 | 0.413 | 0.756 | 0.343 | 0.343 |
| O₂ | kg/hr | 0.00316 | 0.00316 | 0.0825 | 0.0794 | 0.0794 |
| N₂ | kg/hr | 0.345 | 0.345 | 0.604 | 0.259 | 0.259 |
| H₂O | kg/hr | 0.00851 | 0.00851 | 0.00851 | 0 | 0 |
| CO₂ | kg/hr | 0.0508 | 0.0508 | 0.0510 | 0.000208 | 0.000208 |
| CO | kg/hr | 0 | 0 | 0 | 0 | 0 |
| CH₄ | kg/hr | 0 | 0 | 0 | 0 | 0 |
| C₂H₆ | kg/hr | 0 | 0 | 0 | 0 | 0 |
| C₃H₈ | kg/hr | 0 | 0 | 0 | 0 | 0 |
| ARGON | kg/hr | 0.00585 | 0.00585 | 0.0103 | 0.00440 | 0.00440 |
| Mass Fractions | | | | | | |
| O₂ | | 0.00764 | 0.00764 | 0.109 | 0.231 | 0.231 |
| N₂ | | 0.835 | 0.835 | 0.799 | 0.755 | 0.755 |
| H₂O | | 0.0206 | 0.0206 | 0.0113 | 0 | 0 |
| CO₂ | | 0.123 | 0.123 | 0.675 | 0.000608 | 0.000608 |
| CO | | 0 | 0 | 0 | 0 | 0 |
| CH₄ | | 0 | 0 | 0 | 0 | 0 |
| C₂H₆ | | 0 | 0 | 0 | 0 | 0 |
| C₃H₈ | | 0 | 0 | 0 | 0 | 0 |
| ARGON | | 0.0142 | 0.0142 | 0.0136 | 0.0128 | 0.0128 |
| Stream Number | | 344 | 346 | 348 | 352 | 354 |
| Temperature | C. | 750 | 25.0 | 25.0 | 48.1 | 850 |
| Pressure | kPa | 545 | 150 | 150 | 150 | 545 |
| Molar Vapor | | 1.00 | 1.00 | 1.00 | 1.00 | 1.00 |
| Fraction | | | | | | |
| Mass Density | kg/cum | 1.85 | 1.70 | 1.02 | 1.61 | 1.67 |
| Enthalpy Flow | kJ/hr | 264 | −43.0 | −7.65 | −367 | 45.7 |
| Average MW | | 28.97 | 28.01 | 16.81 | 28.63 | 28.73 |
| Mass Flows | kg/hr | 0.343 | 0.0109 | 0.00170 | 0.397 | 0.674 |
| O₂ | kg/hr | 0.0794 | 0 | 0 | 0 | 0 |
| N₂ | kg/hr | 0.259 | 0 | 2.83E−05 | 0.345 | 0.604 |
| H₂O | kg/hr | 0 | 0 | 0 | 0.00486 | 0.00851 |
| CO₂ | kg/hr | 0.000208 | 0 | 2.22E−05 | 0.291 | 0.0510 |
| CO | kg/hr | 0 | 0.0109 | 0 | 0.0109 | 0 |
| CH₄ | kg/hr | 0 | 0 | 0.00154 | 0.00154 | 0 |
| C₂H₆ | kg/hr | 0 | 0 | 9.73E−05 | 9.73E−05 | 0 |
| C₃H₈ | kg/hr | 0 | 0 | 8.92E−06 | 8.92−06 | 0 |
| ARGON | kg/hr | 0.00440 | 0 | 0 | 0.00585 | 0.0103 |
| Mass Fractions | | | | | | |
| O₂ | | 0.231 | 0 | 0 | 0 | 0 |
| N₂ | | 0.755 | 0 | 0.0167 | 0.868 | 0.896 |
| H₂O | | 0 | 0 | 0 | 0.0122 | 0.0126 |
| CO₂ | | 0.000608 | 0 | 0.0131 | 0.0734 | 0.0757 |
| CO | | 0 | 1.00 | 0 | 0.0274 | 0 |
| CH₄ | | 0 | 0 | 0.908 | 0.00388 | 0 |
| C₂H₆ | | 0 | 0 | 0.0573 | 0.000245 | 0 |
| C₃H₈ | | 0 | 0 | 0.00525 | 2.25E−05 | 0 |
| ARGON | | 0.0128 | 0 | 0 | 0.0147 | 0.0152 |
| Stream Number | | 358 | 362 | 366 | 370 | 372 |
| Temperature | C. | 635 | 50.0 | 49.0 | 49.0 | 49.0 |
| Pressure | kPa | 530 | 515 | 150 | 150 | 150 |
| Molar Vapor | | 1.00 | 1.00 | 1.00 | 1.00 | 1.00 |
| Fraction | | | | | | |
| Mass Density | kg/cum | 2.01 | 5.52 | 1.61 | 1.61 | 1.61 |
| Enthalpy Flow | kJ/hr | −125 | −554 | −554 | −316 | −238 |
| Average MW | | 28.73 | 28.73 | 28.73 | 28.73 | 28.73 |
| Mass Flows | kg/hr | 0.674 | 0.674 | 0.674 | 0.385 | 0.289 |
| O₂ | kg/hr | 0 | 0 | 0 | 0 | 0 |
| N₂ | kg/hr | 0.604 | 0.604 | 0.604 | 0.345 | 0.259 |
| H₂O | kg/hr | 0.00851 | 0.00851 | 0.00851 | 0.00486 | 0.00365 |
| CO₂ | kg/hr | 0.0510 | 0.0510 | 0.0510 | 0.0291 | 0.0219 |
| CO | kg/hr | 0 | 0 | 0 | 0 | 0 |
| CH₄ | kg/hr | 0 | 0 | 0 | 0 | 0 |
| C₂H₆ | kg/hr | 0 | 0 | 0 | 0 | 0 |
| C₃H₈ | kg/hr | 0 | 0 | 0 | 0 | 0 |
| ARGON | kg/hr | 0.0103 | 0.0103 | 0.0103 | 0.00585 | 0.00440 |
| Mass Fractions | | | | | | |
| O₂ | | 0 | 0 | 0 | 0 | 0 |
| N₂ | | 0.896 | 0.896 | 0.896 | 0.896 | 0.896 |
| H₂O | | 0.0126 | 0.0126 | 0.0126 | 0.0126 | 0.0126 |
| CO₂ | | 0.0757 | 0.0757 | 0.0757 | 0.0757 | 0.0757 |
| CO | | 0 | 0 | 0 | 0 | 0 |
| CH₄ | | 0 | 0 | 0 | 0 | 0 |
| C₂H₆ | | 0 | 0 | 0 | 0 | 0 |
| C₃H₈ | | 0 | 0 | 0 | 0 | 0 |
| ARGON | | 0.0152 | 0.0152 | 0.0152 | 0.0152 | 0.0152 |

To carry out this simulation the following steps were taken:
A RSTOIC (in AspenPlus) reactor block was used to simulate a combustion chamber (Combustion Chamber 324) which was set to operate adiabatically.
A SEP (in AspenPlus) block was used to simulate an oxygen transport membrane unit (Oxygen Separation Module 302).
A Compressor 338 block was used to simulate a compressor to compress an air input (Air 336) from atmospheric pressure to an assumed pressure of 560 kPa (Air High Pressure 340) stream.
A Compressor 328 block was used to simulate a compressor to compress flue gas (Flue Gas Low Pressure 326) to 545 kPa (Flue Gas High Pressure 330) to have the same pressure as the air input (Air High Temperature 344) entering the oxygen transport membrane unit (Oxygen Separation Module 302).
Heater blocks were used to simulate the process heat exchange (Heat Exchanger 342, Heat Exchanger 356) for heat integration such that the oxygen transport membrane unit (Oxygen Separation Module 302) is being operated at the membrane activation temperature of 850° C.
A flow rate of feed cold air (Air 336) was adjusted such that the following criteria were satisfied:
   (a) flow rate of pure O₂ (stream Permeate Purge 316), which can correspond to stream in O₂ Enriched Permeate Line 118 in FIG. 1, is exactly what is required in the feed stream of the ODH reactor in Example 1, which is 2.08 mol/h.
   (b) CO from the overhead of a CO-column (not shown) (stream CO 346), which can be equivalent to CO produced inside of an ODH reactor, was mixed with natural gas (fuel in Fuel Line 348) to form stream Mixed Gas 352, which can be equivalent to the fuel stream in Fuel Line 152 in FIG. 1, to generate the heat needed to run the oxygen transport membrane, Oxygen Separation Module 302.

In order to control the temperature of the combustion chamber, Combustion Chamber 324, a retentate stream (stream Retentate High Temperature 354) from the retentate side from the oxygen transport membrane unit (Oxygen Separation Module 302), which would correspond with the stream in O₂ Depleted Air Exhaust Line 146 in FIG. 1, was recycled and mixed with fuel going into the combustion chamber, Combustion Chamber 324.

The net temperature of the stream to the oxygen transport membrane unit (Oxygen Separation Module 302), stream Feed to Membrane 334, is 850° C.

### Example 3

### Heat Recovery from Flue Gas

Simulations were developed using AspenPlus® V10. In this simulation, there is no direct mixing of the hot flue gas with the compressed air input going into the oxygen transport membrane unit (Oxygen Separation Module 402) as shown in FIG. 4. The Peng-Robinson equation of state was used. Steam properties were calculated using STEAMNBS. The stream results are shown in Table 4.

TABLE 4
AspenPlus Stream Results for Example 3

| Stream | | | | | | | |
|---|---|---|---|---|---|---|---|
| Number | | 404 | 408 | 410 | 414 | 418 | 422 |
| Temperature | C. | 850 | 850 | 850 | 320 | 40.0 | 39.2 |
| Pressure | kPa | 530 | 110 | 530 | 515 | 500 | 150 |
| Molar Vapor | | 1 | 1 | 1 | 1 | 1 | 1 |
| Fraction | | | | | | | |
| Mass Density | kg/cum | 1.81 | 0.377 | 1.60 | 2.94 | 5.42 | 1.63 |
| Enthalpy | kJ/hr | 56.3 | 56.3 | 198 | 66.4 | 1.59 | 1.59 |
| Flow | | | | | | | |
| Average MW | | 32.00 | 32.00 | 28.16 | 28.16 | 28.16 | 28.16 |
| Mass Flows | kg/hr | 0.0666 | 0.0666 | 0.221 | 0.221 | 0.221 | 0.221 |
| O₂ | kg/hr | 0.0666 | 0.0666 | 0 | 0 | 0 | 0 |
| N₂ | kg/hr | 0 | 0 | 0.217 | 0.217 | 0.217 | 0.217 |
| H₂O | kg/hr | 0 | 0 | 0 | 0 | 0 | 0 |
| CO₂ | kg/hr | 0 | 0 | 175E−04 | 1.75E−04 | 175E−04 | 175E−04 |
| CO | kg/hr | 0 | 0 | 0 | 0 | 0 | 0 |
| CH₄ | kg/hr | 0 | 0 | 0 | 0 | 0 | 0 |
| C₂H₆ | kg/hr | 0 | 0 | 0 | 0 | 0 | 0 |
| C₃H₈ | kg/hr | 0 | 0 | 0 | 0 | 0 | 0 |
| ARGON | kg/hr | 0 | 0 | 3.69E−03 | 3.69E−03 | 3.6E−03 | 3.69E−03 |
| Mass | | | | | | | |
| Fractions | | | | | | | |
| O₂ | | 1.00 | 1.00 | 0 | 0 | 0 | 0 |
| N₂ | | 0 | 0 | 0.983 | 0.983 | 0.983 | 0.983 |
| H₂O | | 0 | 0 | 0 | 0 | 0 | 0 |
| CO₂ | | 0 | 0 | 0.000791 | 0.000791 | 0.000791 | 0.000791 |
| CO | | 0 | 0 | 0 | 0 | 0 | 0 |
| CH₄ | | 0 | 0 | 0 | 0 | 0 | 0 |
| C₂H₆ | | 0 | 0 | 0 | 0 | 0 | 0 |
| C₃H₈ | | 0 | 0 | 0 | 0 | 0 | 0 |
| ARGON | | 0 | 0 | 0.0167 | 0.0167 | 0.0167 | 0.0167 |

| Stream | | | | | | |
|---|---|---|---|---|---|---|
| Number | | 426 | 428 | 432 | 434 | 436 |
| Temperature | C. | 25.0 | 33.8 | 25.0 | 25.0 | 33.1 |
| Pressure | kPa | 150 | 150 | 150 | 150 | 150 |
| Molar Vapor | | 1 | 1 | 1 | 1 | 1 |
| Fraction | | | | | | |
| Mass Density | kg/cum | 1.76 | 1.68 | 1.70 | 1.02 | 1.65 |
| Enthalpy Flow | kJ/hr | −122 | −120 | −43.0 | −38.7 | −202 |
| Average MW | | 29.07 | 28.50 | 28.01 | 16.81 | 28.04 |
| Mass Flows | kg/hr | 0.137 | 0.358 | 0.0109 | 0.00859 | 0.378 |
| O₂ | kg/hr | 0 | 0 | 0 | 0 | 0 |
| N₂ | kg/hr | 0.123 | 0.341 | 0 | 1.43E−04 | 0.341 |
| H₂O | kg/hr | 0 | 0 | 0 | 0 | 0 |
| CO₂ | kg/hr | 1.36E−02 | 1.38E−02 | 0 | 1.13E−04 | 1.39E−02 |
| CO | kg/hr | 0 | 0 | 0.0109 | 0 | 0.0109 |
| CH₄ | kg/hr | 0 | 0 | 0 | 0.00780 | 0.00780 |
| C₂H₆ | kg/hr | 0 | 0 | 0 | 4.92E−04 | 4.92E−04 |
| C₃H₈ | kg/hr | 0 | 0 | 0 | 4.51E−05 | 4.51E−05 |
| ARGON | kg/hr | 0 | 3.69E−03 | 0 | 0 | 3.69E−03 |
| Mass Fractions | | | | | | |
| O₂ | | 0 | 0 | 0 | 0 | 0 |
| N₂ | | 0.900 | 0.951 | 0 | 0.0167 | 0.902 |
| H₂O | | 0 | 0 | 0 | 0 | 0 |
| CO₂ | | 0.0997 | 0.0386 | 0 | 0.01310 | 0.0369 |
| CO | | 0 | 0 | 1.00 | 0 | 0.0288 |
| CH₄ | | 0 | 0 | 0 | 0.908 | 0.0206 |
| C₂H₆ | | 0 | 0 | 0 | 0.0573 | 0.00130 |
| C₃H₈ | | 0 | 0 | 0 | 0.00525 | 1.19E−04 |
| ARGON | | 0 | 0.0103 | 0 | 0 | 0.0098 |
| Stream | | | | | | |
| Number | | 440 | 444 | 448 | 452 | 454 |
| Temperature | C. | 198 | 1016 | 880 | 125 | 25.0 |
| Pressure | kPa | 150 | 130 | 115 | 100 | 100 |
| Molar Vapor | | 1 | 1 | 1 | 1 | 1 |
| Fraction | | | | | | |
| Mass Density | kg/cum | 1.08 | 0.347 | 0.343 | 0.864 | 1.17 |
| Enthalpy Flow | kJ/hr | −107 | −107 | −200 | −674 | −2.62 |
| Average MW | | 28.32 | 28.60 | 28.60 | 28.60 | 28.97 |
| Mass Flows | kg/hr | 0.548 | 0.548 | 0.548 | 0.548 | 0.458 |
| O₂ | kg/hr | 0.0394 | 5.97E−05 | 5.97E−05 | 5.97E−05 | 0.106 |
| N₂ | kg/hr | 0.469 | 0.469 | 0.469 | 0.469 | 0.346 |
| H₂O | kg/hr | 0 | 0.0185 | 0.0185 | 0.0185 | 0 |
| CO₂ | kg/hr | 1.40E−02 | 5.41E−02 | 5.41E−02 | 5.41E−02 | 2.78E−04 |
| CO | kg/hr | 0.0109 | 0 | 0 | 0 | 0 |
| CH₄ | kg/hr | 0.00780 | 0 | 0 | 0 | 0 |
| C₂H₆ | kg/hr | 4.92E−04 | 0 | 0 | 0 | 0 |
| C₃H₈ | kg/hr | 4.51E−05 | 0 | 0 | 0 | 0 |
| ARGON | kg/hr | 5.87E−03 | 5.87E−03 | 5.87E−03 | 5.87E−03 | 5.87E−03 |
| Mass Fractions | | | | | | |
| O₂ | | 0.0719 | 1.09E−04 | 1.09E−04 | 1.09E−04 | 0.231 |
| N₂ | | 0.857 | 0.857 | 0.857 | 0.857 | 0.755 |
| H₂O | | 0 | 0.0337 | 0.0337 | 0.0337 | 0 |
| CO₂ | | 0.0256 | 0.0988 | 0.0988 | 0.0988 | 6.08E−04 |
| CO | | 0.0199 | 0 | 0 | 0 | 0 |
| CH₄ | | 0.0142 | 0 | 0 | 0 | 0 |
| C₂H₆ | | 8.99E−04 | 0 | 0 | 0 | 0 |
| C₃H₈ | | 8.24E−05 | 0 | 0 | 0 | 0 |
| ARGON | | 0.0107 | 0.0107 | 0.0107 | 0.0107 | 0.0128 |
| Stream | | | | | | |
| Number | | 458 | 462 | 466 | 468 | 472 |
| Temperature | C. | 298 | 565 | 565 | 565 | 850 |
| Pressure | kPa | 560 | 545 | 545 | 545 | 530 |
| Molar Vapor | | 1 | 1 | 1 | 1 | 1 |
| Fraction | | | | | | |
| Mass Density | kg/cum | 3.41 | 2.26 | 2.26 | 2.26 | 1.64 |
| Enthalpy Flow | kJ/hr | 125 | 257 | 95.4 | 161 | 255 |
| Average MW | | 28.97 | 28.97 | 28.97 | 28.97 | 28.97 |
| Mass Flows | kg/hr | 0.458 | 0.458 | 0.170 | 0.288 | 0.288 |
| O₂ | kg/hr | 0.106 | 0.106 | 0.0394 | 0.0666 | 0.0666 |
| N₂ | kg/hr | 0.346 | 0.346 | 0.128 | 0.217 | 0.217 |
| H₂O | kg/hr | 0 | 0 | 0 | 0 | 0 |
| CO₂ | kg/hr | 2.78E−04 | 2.78E−04 | 1.03E−04 | 1.75E−04 | 1.75E−04 |
| CO | kg/hr | 0 | 0 | 0 | 0 | 0 |
| CH₄ | kg/hr | 0 | 0 | 0 | 0 | 0 |
| C₂H₆ | kg/hr | 0 | 0 | 0 | 0 | 0 |
| C₃H₈ | kg/hr | 0 | 0 | 0 | 0 | 0 |
| ARGON | kg/hr | 5.87E−03 | 5.87E−03 | 2.18E−03 | 3.69E−03 | 3.69E−03 |
| Mass Fractions | | | | | | |
| O₂ | | 0.231 | 0.231 | 0.231 | 0.231 | 0.231 |
| N₂ | | 0.755 | 0.755 | 0.755 | 0.755 | 0.755 |
| H₂O | | 0 | 0 | 0 | 0 | 0 |
| CO₂ | | 6.08E−04 | 6.08E−04 | 6.08E−04 | 6.08E−04 | 6.08E−04 |
| CO | | 0 | 0 | 0 | 0 | 0 |
| CH₄ | | 0 | 0 | 0 | 0 | 0 |
| C₂H₆ | | 0 | 0 | 0 | 0 | 0 |
| C₃H₈ | | 0 | 0 | 0 | 0 | 0 |
| ARGON | | 0.0128 | 0.0128 | 0.0128 | 0.0128 | 0.0128 |

To carry out this simulation the following steps were taken:
A Compressor 456 block was used to simulate a compressor to compress air from atmospheric pressure to 560 kPa. This was calculated assuming that the pressure of oxygen on the permeate side of an oxygen transport membrane is 110 kPa, which is shown in stream Permeate 408, and with 30 kPa pressure drop from the compressor to the oxygen transport membrane, Oxygen Separation Module 402, which is shown in stream Feed to Membrane 472.
Heater blocks were used to simulate the process heat exchange (Heat Exchanger 460 and Cooler 412; Heat Exchanger 446 and Heat Exchanger 470) for heat integration such that the oxygen transport membrane unit (Oxygen Separation Module 402) is being operated at the oxygen transport membrane activation temperature of 850° C.
A flow rate of feed cold air (Air 454) was adjusted such that the following criteria were satisfied:
   (a) flow rate of pure O₂ (stream Permeate 408) is exactly what is required in the feed stream of an ODH reactor in Example 1, which is 2.08 mol/h.
   (b) CO from the overhead of a CO-column (not shown) (stream CO 432), which can be equivalent to CO produced inside of an ODH reactor, was mixed with natural gas (fuel in Fuel Line 434) to form stream Fuel and Retentate 436, which can be equivalent to the fuel stream in Fuel Line 252 in FIG. 2, to generate the heat needed to run the membrane.
In order to control the temperature of the burner, Combustion Chamber 442, the retentate stream (stream Retentate High Temperature 410) from the oxygen transport membrane unit (Oxygen Separation Module 402) was recycled and mixed with fuel (in Fuel Line 434 and CO 432) going into the combustion chamber. Make-up diluent may be required, which could be CO₂ generated in an ODH Reactor and/or part of the cooled flue gas from the combustion chamber, the flue gas stream in Flue Gas Line 452. The net temperature of the stream to the oxygen transport membrane unit, Feed to Membrane 472, is 850° C.

While the present disclosure has been particularly set forth in terms of specific embodiments thereof, it will be understood in view of the instant disclosure that numerous variations upon the disclosure are now enabled yet reside within the scope of the disclosure. Accordingly, the disclosure is to be broadly construed and limited only by the scope and spirit of the claims now appended hereto.

### INDUSTRIAL APPLICABILITY

The present disclosure relates to a chemical complex for the oxidative dehydrogenation of C2-C4 alkanes into corresponding C2-C4 alkenes.

1. A chemical complex for oxidative dehydrogenation of C2-C4 alkanes, the chemical complex comprising:
an oxidative dehydrogenation reactor,
a quench tower,
an amine wash tower,
a dryer,
a distillation tower,
a combustion chamber, and
an oxygen separation module;
wherein the oxidative dehydrogenation reactor comprises a mixed metal oxide catalyst and is designed to accept an oxygen containing gas and a C2-C4 alkane containing gas, and to produce a product stream comprising a corresponding C2-C4 alkene and one or more of: an unreacted C2-C4 alkane; oxygen; one or more carbon oxides selected from carbon dioxide and carbon monoxide; one or more oxygenates selected from acetic acid, acrylic acid and maleic acid; and water;
wherein the quench tower is adapted to quench the product stream and remove water and soluble oxygenates from the product stream to provide a quenched product stream;
wherein the amine wash tower is adapted to remove carbon dioxide from the quenched product stream to provide a washed product stream;
wherein the dryer is adapted to remove water from the washed product stream to provide a dried product stream;
wherein the distillation tower is adapted to remove C2/C2+ hydrocarbons from the dried product stream to produce an overhead stream comprising C1 hydrocarbons;
wherein the combustion chamber is adapted to receive the overhead stream and a fuel stream and combust the overhead stream, the combustion chamber producing heat and a flue gas at a temperature of about 850° C. to about 1500° C.;
wherein the flue gas is used to provide heat to the oxygen separation module either by introducing the flue gas to the oxygen separation module or by using the flue gas to heat an oxygen containing stream that is introduced to the oxygen separation module;
wherein the oxygen separation module comprises:
an oxygen transport membrane housed inside a sealed vessel and having a retentate side and a permeate side;
a first inlet for introducing the flue gas or an oxygen containing stream, or both into the retentate side;
a second inlet for introducing the flue gas or the oxygen containing stream, or both into the permeate side;
an air inlet for introducing air into the retentate side;
an exhaust stream outlet for discharge of oxygen depleted air and combustion products from the retentate side; and
an outlet stream for removing oxygen enriched gas and combustion products from the permeate side;
wherein the oxygen enriched gas from the permeate side is directed back to the oxidative dehydrogenation reactor to make up at least part of the oxygen containing gas introduced into the oxidative dehydrogenation reactor.

2. The chemical complex of claim 1, wherein the stream comprising oxygen to the combustion chamber comprises at least part of the outlet stream for removing oxygen enriched gas and combustion products from the permeate side.

3. The chemical complex of claim 2, wherein at least part of the flue gas from the combustion chamber is recycled to the oxygen separation module supplying heat, such that the temperature of the oxygen transport membrane is from about 850° C. to about 1500° C.

4. The chemical complex of claim 1, wherein the stream comprising oxygen to the combustion chamber comprises at least part of the oxygen depleted air and combustion products from the retentate side.

5. The chemical complex of claim 4, wherein at least part of the flue gas from the combustion chamber recycled to the oxygen separation module supplying heat, such that the temperature of the oxygen transport membrane is from about 850° C. to about 1500° C.

6. The chemical complex of claim 1, wherein the stream comprising oxygen to the combustion chamber comprises the outlet stream removing oxygen enriched gas and combustion productions from the permeate side, and the stream comprising oxygen to the same or a different combustion chamber comprises the exhaust stream of oxygen depleted air combustion products from the retentate side.

7. The chemical complex of claim 6, wherein at least part of the flue gas from the combustion chamber recycled to the oxygen separation module supplying heat, such that the temperature of the oxygen transport membrane is from about 850° C. to about 1500° C.

8. The chemical complex of claim 1, wherein the temperature of the oxygen transport membrane is from about 850° C. to about 1250° C.

9. The chemical complex of claim 1, wherein the temperature of the oxygen transport membrane is from about 850° C. to about 1000° C.

10. The chemical complex of claim 1, wherein the pressure in the combustion chamber is atmospheric to about 700 kPag.

11. The chemical complex of claim 1, wherein the mixed metal oxide catalyst comprises one or more compounds selected from:
i) catalysts of the formula:
MoₐV_{b}Te_{c}Nb_{d}PdₑO_{f}
wherein a, b, c, d, e and f are the relative atomic amounts of the elements Mo, V, Te, Nb, Pd and O, respectively; and when a=1, b=0.01 to 1.0, c=0 to 1.0, d=0 to 1.0, 0≤e≤0.10 and f is a number to satisfy the valence state of the catalyst;
ii) catalysts of the formula:
Ni_{g}AₕBᵢDⱼO_{f}
wherein: g is a number from 0.6 to 0.8; h is a number from 0.04 to 0.9; i is a number from 0 to 0.5; j is a number from 0 to 0.5; and f is a number to satisfy the valence state of the catalyst; A is selected from the group consisting of Ti, Ta, V, Nb, Hf, W, Y, Zn, Zr, Si and Al or mixtures thereof; B is selected from the group consisting of La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg, and mixtures thereof; D is selected from the group consisting of Ca, K, Mg, Li, Na, Sr, Ba, Cs, and Rb and mixtures thereof; and O is oxygen;
iii) catalysts of the formula:
MoₐEₖGₗO_{f}
wherein: E is selected from the group consisting of Ba, Be, Ca, Cr, Mn, Nb, Ta, Ti, Te, V, W and mixtures thereof; G is selected from the group consisting of Al, Bi, Ce, Co, Cu, Fe, K, Mg, V, Ni, P, Pb, Sb, Si, Sn, Ti, U, and mixtures thereof; a=1; k is 0 to 2; l=0 to 2, with the proviso that the total value of 1 for Co, Ni, Fe and mixtures thereof is less than 0.5; and f is a number to satisfy the valence state of the catalyst;
iv) catalysts of the formula:
VₘMoₙNbₒTeₚMe_{q}O_{f}
wherein: Me is a metal selected from the group consisting of Ta, Ti, W, Hf, Zr, Sb and mixtures thereof; m is from 0.1 to 3; n is from 0.5 to 1.5; o is from 0.001 to 3; p is from 0.001 to 5; q is from 0 to 2; and f is a number to satisfy the valence state of the catalyst;
v) catalysts of the formula:
MoₐVᵣXₛYₜZᵤMᵥO_{f}
wherein: X is at least one of Nb and Ta; Y is at least one of Sb and Ni; Z is at least one of Te, Ga, Pd, W, Bi and Al; M is at least one of Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t=0.001 to 1.0; u=0.001 to 0.5; v=0.001 to 0.3; and f is a number to satisfy the valence state of the catalyst;
vi) a mixed metal oxide having the empirical formula:
Mo_{6.5-7.0}V₃O_{d}
wherein d is a number to at least satisfy the valence of the metals in the catalyst; and
vii) a mixed metal oxide having the empirical formula:
Mo_{6.25-7.25}V₃O_{d}
wherein d is a number to at least satisfy the valence of the metals in the catalyst.

12. The chemical complex of claim 1, wherein the mixed metal oxide catalyst comprises a compound selected from:
Mo₁V_{0.1-1}Nb_{0.1-1}Te_{0.01-0.2}X_{0-0.2}O_{f}
wherein X is selected from Pd, Sb Ba, Al, W, Ga, Bi, Sn, Cu, Ti, Fe, Co, Ni, Cr, Zr, Ca and oxides and mixtures thereof, and f is a number to satisfy the valence state of the catalyst.

13. The chemical complex of claim 1, wherein the C2-C4 alkane is ethane.

14. An oxygen separation module, comprising:
an oxygen transport membrane housed inside a sealed vessel and having a retentate side and a permeate side;
a first inlet for introducing a flue gas or an oxygen containing stream, or both into the retentate side;
a second inlet for introducing the flue gas or the oxygen containing stream, or both into the permeate side;
an air inlet for introducing air into the retentate side;
an exhaust stream outlet for discharge of oxygen depleted air and combustion products from the retentate side; and
an outlet stream for removing oxygen enriched gas and combustion products from the permeate side;
wherein the oxygen enriched gas from the permeate side is directed back to an oxidative dehydrogenation reactor to make up at least part of an oxygen containing gas introduced into the oxidative dehydrogenation reactor.

15. The oxygen separation module of claim 14, wherein the flue gas is provided by a combustion chamber that is adapted to combust a stream comprising C1 hydrocarbons; wherein the flue gas is at a temperature of about 850° C. to about 1500° C.; and wherein the flue gas is used to provide heat to the oxygen separation module by introducing the flue gas to the oxygen separation module, or by using the flue gas to heat an oxygen containing stream that is introduced to the oxygen separation module, or both.

16. The oxygen separation module of claim 15, wherein the combustion chamber is fed at least part of the outlet stream for removing oxygen enriched gas and combustion products from the permeate side.

17. The oxygen separation module of claim 14, wherein the temperature of the oxygen transport membrane is from about 850° C. to about 1500° C.

18. The oxygen separation module of claim 14, wherein the temperature of the oxygen transport membrane is from about 850° C. to about 1500° C.

19. The oxygen separation module of claim 14, wherein the temperature of the oxygen transport membrane is from about 850° C. to about 1250° C.

20. The oxygen separation module of claim 14, wherein the temperature of the oxygen transport membrane is from about 850° C. to about 1000° C.
